(19) United States
(12) Patent Application Publication
(10) Pub. No.: US 2024/0393288 A1
(43) Pub. Date: Nov. 28, 2024

(54) Electrochemical Aptamer Sensors With Stable Blocking Layers, Rapid Electron Transfer And Robust Antifouling Properties

(71) Applicant: University Of Cincinnati, Cincinnati, OH (US)

(72) Inventor: Jason Charles Heikenfeld, Cincinnati, OH (US)

(73) Assignee: University Of Cincinnati, Cincinnati, OH (US)

(21) Appl. No.: 18/694,980

(22) PCT Filed: Sep. 23, 2022
(86) PCT No.: PCT/US2022/044512

§ 371 Date: Mar. 24, 2024

Related U.S. Application Data
(60) Provisional application No. 63/248,016, filed on Sep. 24, 2021.
(60) Provisional application No. 63/282,440, filed on Nov. 23, 2021.
(60) Provisional application No. 63/307,215, filed on Feb. 7, 2022.
(60) Provisional application No. 63/339,196, filed on May 6, 2022.

(51) Int. Cl. G01N 27/327; G01N 33/53; G01N 33/543

(52) U.S. Cl. CPC G01N 27/3277; G01N 27/3276; G01N 33/5308; G01N 33/5438

(57) ABSTRACT

The present invention relates to a device for continuous sensing of at least one analyte in a test fluid that is resistant at least one of desorption of a first plurality of molecules, including a plurality of aptamers and a blocking layer, and fouling during use of the device when exposed to temperatures greater than or equal to 30° C. for at least 3 days. Another aspect of the invention is a method of providing fouling resistance to an aptamer sensor device wherein the blocking layer and aptamer are resistant to at least one of desorption from the electrode and fouling during use of the aptamer sensor when exposed to temperatures greater than or equal to 30° C. for at least 3 days.

### CROSS-REFERENCE TO RELATED APPLICATIONS

This application claims priority to, and the benefit of the filing date of, U.S. Application Ser. No. 63/307,215, titled “Electrochemical Aptamer Sensors with Stable Blocking Layers, Rapid Electron Transfer and Robust Antifouling Properties” which was filed on Feb. 7, 2022 on—the disclosure of which is incorporated by reference herein in its entirety; and claims priority to, and the benefit of the filing date of, U.S. Application Ser. No. 63/339,196, titled “Electrochemical Aptamer Sensors with Stable Blocking Layers, Rapid Electron Transfer and Robust Antifouling Properties” which was filed on May 6, 2022—the disclosure of which is incorporated by reference herein in its entirety, and the benefit of the filing date of, U.S. Ser. No. 63/248,016, titled “Electrochemical Aptamer Sensor Monolayer Incubation with Improved Stability,” which was filed Sep. 24, 2021, the disclosure of which is hereby incorporated herein by reference in its entirety, and the benefit of the filing date of, U.S. Application Ser. No. 63/282,440, titled “Electrochemical Aptamer Sensors with Non-monolayer Blocking Layers,” which was filed on Nov. 23, 2021—the disclosure of which is incorporated by reference herein in its entirety.

### FIELD OF THE INVENTION

This invention relates generally to aptamer sensors, and more specifically to aptamer sensors with improved longevity of operation.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical aptamer sensors can identify the presence and/or concentration of an analyte of interest via the use of an aptamer sequence that specifically binds to the analyte of interest. These sensors include aptamers attached to an electrode, wherein each of the aptamers has a redox active molecule (redox tag) attached thereto. The redox couple can transfer electrical charge to or from the electrode. When an analyte binds to the aptamer, the aptamer changes shape, bringing the redox couple closer to or further from, on average, the electrode. This results in a measurable change in electrical current that can be translated to a measure of presence or concentration of the analyte. When used in this manner, then, aptamers are an example of an affinity-based biosensor.

A major unresolved challenge for aptamer sensors and other affinity-based biosensors (particularly those where the aptamers are bonded to the working electrode) is the lifetime of the sensors, especially for applications where continuous operation is required (“continuous” referring to multiple measurements over time by the same device). Such aptamer sensors are susceptible to degradation due to, among other things, desorption of the aptamers themselves from the electrode, and/or desorption of the blocking layer molecules (such as mercaptohexanol) from the electrode. The aptamers and the blocking molecules together form a monolayer which can be referred to as a sensing monolayer. The blocking layer portion of the sensing monolayer (1) ensures that the aptamer conformation change when binding to an analyte is not physically hindered by foulants, and (2) reduces electrical background current (including oxygen reduction current), which would otherwise wash-out the measured signal from the aptamer and redox tag.

Current methods of fabrication of these devices uses a very simple and convenient approach of forming a partial monolayer of aptamer by thiol bonding to a gold electrode via incubation of the electrode in solution including aptamer(s), followed by forming a more complete monolayer including the blocking molecule such as mercaptohexanol (via incubation of the electrode in mercaptohexanol solution). This process is quite fortuitous for researchers because not only does a monolayer of mercaptohexanol reduce background current, but mercaptohexanol monolayers as-typically-formed have at least one feature such as defects, for example, which allow for electron transfer between the redox tag and the electrode, these defects being few and/or small enough to minimize oxygen reduction current and other major sources of background current. Furthermore, mercaptohexanol monolayers have adequate defects for electron transfer to support a zero gain frequency that allows two frequency or comparable self-calibration techniques. Lastly, mercaptohexanol has enough surface fouling resistance to allow for short-term in-lab experiments in biofluids such as blood or serum.

Therefore, researchers have had at their disposal a very ‘convenient’ way to make aptamer sensors for research applications. However, most aptamer researchers have not historically been motivated to address longevity of aptamer sensors, and the same monolayer approach that is so convenient is also inherently fragile as the monolayer is able to desorb over time. Part of this cause for desorption is that each portion of the monolayer is a single molecule that has a single bond to the electrode, and statistically or energetically breaking one of these bonds with the electrode is not that difficult with conventional monolayer chemistries, especially at elevated temperatures such as body temperature. Multiple bonds to the gold could alleviate this challenge, but also may lack the tight packing density required for a low background current during measurement. According to leading experts in the 2022 review article, see Shaver, et al., “The challenge of long-term stability for nucleic acid-based electrochemical sensors,” Current Opinion in Electrochemistry (2022), 32:100902 (https://doi.org/10.1016/j.coelec.2021.100902), “Unfortunately, these chemistries desorb over time when exposed to environmental or experimental factors like, for example, dry air, high temperature, voltage pulsing, and biological fluids. This desorption process simultaneously removes sensing moieties and passivating thiols from the electrode surface, prohibiting their deployment for more than a few hours.” Clearly, even to the experts in the field, aptamer sensor longevity remains an unresolved problem with no obvious solutions for achieving sensor longevity for multiple days or weeks. Even as alternate methods are developed for extending longevity of blocking layers, these methods must also support proper electron-transfer for sensor signaling, ideally allow use of one or more calibration-free methods of operation, and prevent over-fouling that otherwise would inhibit movement of the aptamer and therefore proper signaling of the sensor. Novel approaches for electrochemical aptamer sensors which reduce or eliminate these drawbacks could provide significant benefits in longevity and ideally would still operate with a robust redox tag signal, enable two frequency operation for self-calibration, and other aspects that make the sensors attractive for biosensing applications.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

One aspect of the present invention is directed to device for continuous sensing of at least one analyte in a sample fluid, including: an electrode having an electrode surface; a first plurality of molecules including: a plurality of aptamers comprising attached redox tags, wherein the attached redox tags provide electron transfer with the electrode; and a blocking layer formed on the electrode, wherein the blocking layer includes a blocking layer surface and a plurality of features supporting the electron transfer between the electrode and the redox tags; and wherein the first plurality of molecules, when exposed to temperatures greater than or equal to 30° C., is resistant to at least one of desorption from the electrode and fouling during use of the device for at least 3 days. In one such embodiment, the first plurality of molecules is resistant to at least one of desorption from the electrode and fouling during use of the device when exposed temperatures greater than or equal to 30° C. and less than or equal to 47° C. In one such embodiment, the blocking layer includes a plurality of mercaptooctanol molecules.

The device may include a blocking layer that includes a material having a total binding energy. In one such embodiment, the blocking layer includes a material having a total binding energy equal to or more negative than −3.05 eV. In one such embodiment, the blocking layer includes a material having a total binding energy equal to or more negative than −3.1 eV.

The device may have a portion of the first plurality of molecules that are weakly bonded to the electrode surface. In one such embodiment, less than or equal to 40% of the first plurality of molecules are weakly bonded to the electrode surface. In one such embodiment, less than or equal to 20% of the first plurality of molecules are weakly bonded to the electrode surface. In one such embodiment, less than or equal to 10% of the first plurality of molecules are weakly bonded to the electrode surface. In one such embodiment, less than or equal to 5% of the first plurality of molecules are weakly bonded to the electrode surface.

The device may include a gold electrode that has an average slope roughness. In one such embodiment, the electrode has an average slope roughness of at least 0.5%. In one such embodiment, the electrode has an average slope roughness of at least 1%. In one such embodiment, the electrode has an average slope roughness of at least 2%. In one such embodiment, the electrode has an average slope roughness of at least 5%. In one such embodiment, the electrode has an average slope roughness of at least 10%. In one such embodiment, the electrode has an average slope roughness of at least 20%. In one such embodiment, the electrode has an average slope roughness of at least 40%.

The device may include features such that a majority of the plurality of features in the blocking layer are defects having a certain size. In one such embodiment, the defects are less than 0.3 nm in size. In a further embodiment thereof, a majority of the plurality of defects are at least 0.01 nm in size.

The device may include features such that a majority of the plurality of features in the blocking layer are defects each having a size represented as a fractional area of a surface area of the electrode. In one such embodiment, each of the defects has a fractional area that is less than or equal to 0.2. In one such embodiment, each of the defects has a fractional area that is less than or equal to 0.1. In one such embodiment, each of the defects has a fractional area that is less than or equal to 0.05. In one such embodiment, each of the defects has a fractional area that is less than or equal to 0.02. In one such embodiment, each of the defects has a fractional area that is greater than or equal to 0.001. In one such embodiment, each of the defects has a fractional area that is greater than or equal to 0.002. In one such embodiment, each of the defects has a fractional area that is greater than or equal to 0.005. In one such embodiment, each of the defects has a fractional area that is greater than or equal to 0.01.

The device may include a blocking layer having a terminus moiety that can reduce fouling. In one such embodiment, the terminus moiety has a size less than or equal to 50 A°. In one such embodiment, the terminus moiety has a size less than or equal to 30 A°. In one such embodiment, the terminus moiety has a size less than or equal to 20 A°. In one such embodiment, the terminus moiety has a size less than or equal to 10 A°.

The device may include a blocking layer including a plurality of blocking molecules each having a terminus moiety, wherein the terminus moiety reduces fouling, and wherein the plurality of blocking molecules self-assembles into defect-free ordered groups containing a number of blocking molecules. In one such embodiment, the plurality of blocking molecules self-assembles in defect-free ordered groups containing at least 10 blocking molecules. In one such embodiment, the plurality of blocking molecules self-assembles in defect-free ordered groups containing at least 20 blocking molecules. In one such embodiment, the plurality of blocking molecules self-assembles in defect-free ordered groups containing at least 50 blocking molecules.

The device may include a blocking layer including a second plurality of molecules each having a terminus moiety that is a highly hydrophilic end group. In one such embodiment, the highly hydrophilic end group is a hydroxyl group. In one such embodiment, the highly hydrophilic end group is a phosphatidylcholine group. In one such embodiment, the highly hydrophilic end group is a zwitterionic group. In one such embodiment, the highly hydrophilic end group is a polyethylene group.

The device may include a blocking layer that is formed from a metal or a semiconductor oxide. In one such embodiment, the blocking layer is formed from a metal. In one such embodiment, the blocking layer is formed from a semiconductor oxide. In a further embodiment, the blocking layer is silicon dioxide.

In one embodiment, the blocking layer is a monolayer blocking layer. In one embodiment, the blocking layer is a non-monolayer blocking layer.

In one embodiment, the blocking layer includes a plurality of blocking molecules that are bonded together. In a further embodiment, the plurality of blocking molecules are chemically bonded to each other.

The device may further include an anti-fouling layer. In one such embodiment, the anti-fouling layer is configured to cover a majority of the blocking layer. In a further embodiment, the anti-fouling layer is formed from a plurality of molecules that are bonded to each other. In an even further embodiment, the anti-fouling layer is formed from a plurality of molecules that are chemically bonded to each other.

The blocking layer may include a plurality of amphiphilic molecules having a head group configured to prevent fouling, a polymer chain, and an anchor group. In one such embodiment, the head group is a zwitter ionic group. In one such embodiment, the head group is a polyethylene group. In one such embodiment, the head group is a phosphatidylcholine group. In one such embodiment, the head group is a hydroxyl group. In one such embodiment, the polymer chain is a polyalkane chain. In one such embodiment, the polymer chain is a polyalkene chain. In one such embodiment, the polymer chain is a polyethylene glycol chain. In one such embodiment, the polymer chain is a polypropylene chain. In one such embodiment, the anchor group is a thiol anchor group. In one such embodiment, the anchor group is a silane anchor group. In one such embodiment, the anchor group is a phosphate anchor group.

In one embodiment, the anchor group is bound to a surface selected from the group consisting of the electrode surface and the blocking layer surface and the anchor group is selected based on the surface it is bound to. In a further embodiment, the pair of the surface and the anchor group is a gold electrode surface and a thiol anchor group. In a different further embodiment, the pair of the surface and the anchor group is a silver electrode surface and a thiol anchor group. In yet another different further embodiment, the pair of the surface and the anchor group is a glass electrode surface and a silane anchor group. In a different further embodiment still, the pair of the surface and the anchor group is a silicon electrode surface and a silane anchor group. In another different further embodiment, the pair of the surface and the anchor group is a metal oxide blocking layer surface and a silane anchor group. In another different further embodiment, the pair of the surface and the anchor group is a metal oxide blocking layer surface and a phosphate anchor group.

The device may further include a protective membrane layer. In one such embodiment, the protective membrane layer is a hydrogel. In one such embodiment, the protective membrane layer is a membrane. In a further embodiment, the protective membrane layer is a cross-linked polybetaine membrane.

The device may be capable of measuring a plurality of measurements when placed in a test fluid, wherein one or more of the measurements change in response to binding of the analyte with the plurality of aptamers. In one such embodiment, the plurality of measurements includes a redox current. In one such embodiment, the plurality of measurements includes a background current. In one such embodiment, the plurality of measurements includes a signal gain. In one such embodiment, the plurality of measurements includes a frequency response. In one such embodiment, the plurality of measurements includes a sensor signal. In one such embodiment, the plurality of measurements includes a redox current, a background, a signal gain, a frequency response, and a sensor signal.

A device capable of measuring a plurality of measurements as described above may have an initial sensor gain when initially placed in the test fluid, the sensor signal gain decreasing by less than or equal to 4 times the initial sensor signal gain.

A device capable of measuring a plurality of measurements as described above may further include at least one of an anti-fouling layer or protective membrane layer which preserves greater than or equal to 90% of the sensor signal when compared to an initial sensor signal for greater than or equal to 2 hours of placement in the test fluid.

A device capable of measuring a plurality of measurements as described above may further include at least one of an anti-fouling layer or protective membrane which preserves a percentage of sensor signal for a given time period. In one such embodiment, greater than or equal to 50% of the sensor signal and signal gain is preserved for at least 3 days. In one such embodiment, greater than or equal to 50% of the sensor signal and signal gain is preserved for at least 4 days. In one such embodiment, greater than or equal to 50% of the sensor signal and signal gain is preserved for at least 5 days. In one such embodiment, greater than or equal to 80% of the sensor signal and signal gain is preserved for at least 3 days. In one such embodiment, greater than or equal to 80% of the sensor signal and signal gain is preserved for at least 4 days. In one such embodiment, greater than or equal to 80% of the sensor signal and signal gain is preserved for at least 5 days.

A device capable of measuring a plurality of measurements as described above may be further capable of measuring a zero frequency response and an initial zero frequency response when the device is initially placed in the test fluid, wherein the zero frequency response shifts by a percentage over a given time period. In one such embodiment, the zero gain frequency shifts by less than or equal to 5% after at least 3 days. In one such embodiment, the zero gain frequency shifts by less than or equal to 10% after at least 3 days. In one such embodiment, the zero gain frequency shifts by less than or equal to 20% after at least 3 days. In one such embodiment, the zero gain frequency shifts by less than or equal to 40% after at least 3 days. In one such embodiment, the zero gain frequency shifts by less than or equal to 80% after at least 3 days. In one such embodiment, the zero gain frequency shifts by less than or equal to 5% after at least 4 days. In one such embodiment, the zero gain frequency shifts by less than or equal to 10% after at least 4 days. In one such embodiment, the zero gain frequency shifts by less than or equal to 20% after at least 4 days. In one such embodiment, the zero gain frequency shifts by less than or equal to 40% after at least 4 days. In one such embodiment, the zero gain frequency shifts by less than or equal to 80% after at least 4 days. In one such embodiment, the zero gain frequency shifts by less than or equal to 5% after at least 5 days. In one such embodiment, the zero gain frequency shifts by less than or equal to 10% after at least 5 days. In one such embodiment, the zero gain frequency shifts by less than or equal to 20% after at least 5 days. In one such embodiment, the zero gain frequency shifts by less than or equal to 40% after at least 5 days. In one such embodiment, the zero gain frequency shifts by less than or equal to 80% after at least 5 days.

In further embodiments thereof, the zero gain frequency may be greater than or equal to a given value. In one such further embodiments, the zero gain frequency is greater than or equal to 2 Hz. In one such further embodiments, the zero gain frequency is greater than or equal to 5 Hz. In one such further embodiments, the zero gain frequency is greater than or equal to 10 Hz. In one such further embodiments, the zero gain frequency is greater than or equal to 20 Hz. In one such further embodiments, the zero gain frequency is greater than or equal to 50 Hz. In one such further embodiments, the zero gain frequency is greater than or equal to 100 Hz.

A device capable of measuring a plurality of measurements as described above may have the sensor signal decrease by a value over a given time period. In one such embodiment, the sensor signal decreases by less than or equal to 5% for at least 3 days. In one such embodiment, the sensor signal decreases by less than or equal to 10% for at least 3 days. In one such embodiment, the sensor signal decreases by less than or equal to 20% for at least 3 days. In one such embodiment, the sensor signal decreases by less than or equal to 40% for at least 3 days. In one such embodiment, the sensor signal decreases by less than or equal to 5% for at least 4 days. In one such embodiment, the sensor signal decreases by less than or equal to 10% for at least 4 days. In one such embodiment, the sensor signal decreases by less than or equal to 20% for at least 4 days. In one such embodiment, the sensor signal decreases by less than or equal to 40% for at least 4 days. In one such embodiment, the sensor signal decreases by less than or equal to 5% for at least 5 days. In one such embodiment, the sensor signal decreases by less than or equal to 10% for at least 5 days. In one such embodiment, the sensor signal decreases by less than or equal to 20% for at least 5 days. In one such embodiment, the sensor signal decreases by less than or equal to 40% for at least 5 days.

A device capable of measuring a plurality of measurements as described above may be further capable of measuring an oxygen reduction current of −0.4 V compared to a sealed Ag/AgCl reference and an initial oxygen current when initially placed in the test fluid, wherein the oxygen reduction current contributes to and increases the background current by a percentage for a time period. In one such embodiment, the oxygen reduction current increases the background current by less than or equal to 5% for at least 3 days. In one such embodiment, the oxygen reduction current increases the background current by less than or equal to 10% for at least 3 days. In one such embodiment, the oxygen reduction current increases the background current by less than or equal to 30% for at least 3 days. In one such embodiment, the oxygen reduction current increases the background current by less than or equal to 5% for at least 4 days. In one such embodiment, the oxygen reduction current increases the background current by less than or equal to 10% for at least 4 days. In one such embodiment, the oxygen reduction current increases the background current by less than or equal to 30% for at least 4 days. In one such embodiment, the oxygen reduction current increases the background current by less than or equal to 5% for at least 5 days. In one such embodiment, the oxygen reduction current increases the background current by less than or equal to 10% for at least 5 days. In one such embodiment, the oxygen reduction current increases the background current by less than or equal to 30% for at least 5 days.

A device capable of measuring a plurality of measurements as described above may be further capable of measuring an initial background current when initially placed in the test fluid, the background current increasing by a percentage for a time period. In one such embodiment, the background current increases by less than or equal to 10% for at least 3 days. In one such embodiment, the background current increases by less than or equal to 30% for at least 3 days. In one such embodiment, the background current increases by less than or equal to 50% for at least 3 days. In one such embodiment, the background current increases by less than or equal to 10% for at least 4 days. In one such embodiment, the background current increases by less than or equal to 30% for at least 4 days. In one such embodiment, the background current increases by less than or equal to 50% for at least 4 days. In one such embodiment, the background current increases by less than or equal to 10% for at least 5 days. In one such embodiment, the background current increases by less than or equal to 30% for at least 5 days. In one such embodiment, the background current increases by less than or equal to 50% for at least 5 days.

A device capable of measuring a plurality of measurements as described above may have an initial signal loss of less than 60% when operating in the test fluid for one day, wherein the device is capable of providing sensor operation for at least 4 days.

A device capable of measuring a plurality of measurements as described above may be capable of implementing two or more frequency calibration free operation when measuring one or more of the measurements.

A device capable of measuring a plurality of measurements as described above may have a background current that increases by a percentage after one day. In one such embodiment, the background current increases by less than or equal to 10% per day. In one such embodiment, the background current increases by less than or equal to 5% per day. In one such embodiment, the background current increases by less than or equal to 2% per day.

A device capable of measuring a plurality of measurements as described above may further include a zero-gain frequency. In one such embodiment, the zero-gain frequency is greater than or equal to 2 Hz. In one such embodiment, the zero-gain frequency is greater than or equal to 5 Hz. In one such embodiment, the zero-gain frequency is greater than or equal to 10 Hz. In one such embodiment, the zero-gain frequency is greater than or equal to 20 Hz. In one such embodiment, the zero-gain frequency is greater than or equal to 50 Hz. In one such embodiment, the zero-gain frequency is greater than or equal to 100 Hz.

A device capable of measuring a plurality of measurements as described above may, after operating for greater than or equal to 4 days, have a fouling resistance percentage of the fouling resistance of mercaptooctanol after 24 hours of operation as measured by the amount of signal decrease over time due to fouling. In one such embodiment, the fouling resistance is greater than or equal to 20%. In one such embodiment, the fouling resistance is greater than or equal to 50%. In one such embodiment, the fouling resistance is greater than or equal to 75%. In one such embodiment, the fouling resistance is greater than or equal to 90%.

A device capable of measuring a plurality of measurements as described above may, after one day of operation, have a fouling-induced signal loss. In one such embodiment, the fouling induced signal loss is less than or equal to 10% per day. In one such embodiment, the fouling induced signal loss is less than or equal to 5% per day. In one such embodiment, the fouling induced signal loss is less than or equal to 2% per day. In one such embodiment, the fouling induced signal loss is less than or equal to 1% per day.

A device capable of measuring a plurality of measurements as described above may further include a sensor accuracy, wherein the sensor accuracy is maintained within a range over 4 days of operation. In one such embodiment, the sensor accuracy is less than or equal to +/−60%. In one such embodiment, the sensor accuracy is less than or equal to +/−40%. In one such embodiment, the sensor accuracy is less than or equal to +/−20%.

A device capable of measuring a plurality of measurements as described above may, after one day of operation, exhibit a change in electron transfer rates. In one such embodiment, the electron transfer rate changes by less than or equal to 10% per day. In one such embodiment, the electron transfer rate changes by less than or equal to 5% per day. In one such embodiment, the electron transfer rate changes by less than or equal to 2% per day.

A device capable of measuring a plurality of measurements as described above may have a zero-gain frequency wherein, after one day of operation, the device exhibits a change in zero-gain frequency. In one such embodiment, the zero-gain frequency changes by less than or equal to 10% per day. In one such embodiment, the zero-gain frequency changes by less than or equal to 5% per day. In one such embodiment, the zero-gain frequency changes by less than or equal to 2% per day.

A device capable of measuring a plurality of measurements as described above may, after one day of operation, exhibit a change in signal response to an analyte measured as a percent signal gain. In one such embodiment, the change in signal response is less than or equal to 10% per day. In one such embodiment, the change in signal response is less than or equal to 5% per day. In one such embodiment, the change in signal response is less than or equal to 2% per day. In one such embodiment, the change in signal response is less than or equal to 1% per day.

A device capable of measuring a plurality of measurements as described above may have a loss in signal gain after 3 days of operation. In one such embodiment, the loss in signal gain is less than or equal to 30%. In one such embodiment, the loss in signal gain is less than or equal to 20%. In one such embodiment, the loss in signal gain is less than or equal to 10%. In one such embodiment, the loss in signal gain is less than or equal to 5%.

A device capable of measuring a plurality of measurements as described above may have a test fluid that is serum. In another embodiment, the test fluid is interstitial fluid.

In one embodiment, a negative voltage is applied to the electrode, and wherein the electrode has a negative absolute voltage limit. In a further embodiment, the negative absolute voltage limit is applied to the electrode for a percentage of the time the sensor is in use. In one such embodiment, the negative absolute voltage limit is applied to the electrode for at least 100% of the time the sensor is in use. In one such embodiment, the negative absolute voltage limit is applied to the electrode for at least 90% of the time the sensor is in use. In one such embodiment, the negative absolute voltage limit is applied to the electrode for at least 50% of the time the sensor is in use. In one such embodiment, the negative absolute voltage limit is applied to the electrode for at least 20% of the time the sensor is in use. In one such embodiment, the negative absolute voltage limit is applied to the electrode for at least 10% of the time the sensor is in use. In one such embodiment, the negative absolute voltage limit is applied to the electrode for at least 5% of the time the sensor is in use. In one such embodiment, the negative absolute voltage limit is applied to the electrode for at least 1% of the time the sensor is in use.

In one embodiment, a negative voltage is applied to the electrode, and wherein the electrode has a negative average voltage. In a further embodiment, the negative average voltage is applied to the electrode for a percentage of the time the sensor is in use. In one such embodiment, the negative average voltage is applied to the electrode for at least 100% of the time the sensor is in use. In one such embodiment, the negative average voltage is applied to the electrode for at least 90% of the time the sensor is in use. In one such embodiment, the negative average voltage is applied to the electrode for at least 50% of the time the sensor is in use. In one such embodiment, the negative average voltage is applied to the electrode for at least 20% of the time the sensor is in use. In one such embodiment, the negative average voltage is applied to the electrode for at least 10% of the time the sensor is in use. In one such embodiment, the negative average voltage is applied to the electrode for at least 5% of the time the sensor is in use. In one such embodiment, the negative average voltage is applied to the electrode for at least 1% of the time the sensor is in use.

Another aspect of the invention is directed to a method of providing fouling resistance to an aptamer sensor, the aptamer sensor including an electrode having an electrode surface and an aptamer having a redox tag, the method comprising: binding a plurality of molecules to the electrode to form a blocking layer having a blocking layer surface, wherein the blocking layer has a plurality of features supporting electron transfer between the electrode and the redox tag, and wherein the blocking layer and the aptamer are resistant to at least one of desorption from the electrode and fouling during use of the sensor when exposed to test fluid and temperatures greater than or equal to 30° C. for at least 3 days; and wherein the aptamer is attached to a surface selected from the group consisting of the electrode surface and the blocking layer surface. In a further embodiment, the blocking layer and aptamer are resistant to at least one of desorption from the electrode and fouling during use of the sensor when exposed to test fluid and temperatures greater than or equal to 30° C. and less than or equal to 47° C.

In one such embodiment, each of the plurality of blocking molecules include an anchor group. In a further embodiment, the anchor group is a thiol. In an even further embodiment, binding the plurality of molecules to form the blocking layer includes weakly binding a percentage of the plurality of blocking molecules to the electrode surface. In one such even further embodiment, less than or equal to 40% of the plurality of blocking molecules are weakly bound. In one such even further embodiment, less than or equal to 20% of the plurality of blocking molecules are weakly bound. In one such even further embodiment, less than or equal to 10% of the plurality of blocking molecules are weakly bound. In one such even further embodiment, less than or equal to 5% of the plurality of blocking molecules are weakly bound.

In one embodiment, the plurality of blocking molecules includes mercaptooctanol.

The method may further include roughening the electrode surface prior to binding the plurality of blocking layer molecules. In a further embodiment thereof, the electrode surface is roughened to have an average slope roughness of less than or equal to 40%. In another further embodiment, the electrode surface is roughened to have an average slope roughness of less than or equal to 0.5%. In yet another further embodiment, the electrode surface is mechanically roughened. In still another further embodiment, the electrode surface is roughened by depositing a metal onto the electrode surface at a rate greater than or equal to 10 nm/min.

The method may further include binding at least one of an anti-fouling layer or a protective membrane to the aptamer sensor. In a further embodiment, the protective membrane is placed above the blocking layer surface. In another further embodiment, the anti-fouling layer or the protective membrane to the electrode. In yet another further embodiment, the anti-fouling layer or protective membrane is bound to the blocking layer.

In still another further embodiment, the anti-fouling layer or the protective membrane comprises a second plurality of molecules. In an even further embodiment thereof, the method further includes crosslinking the second plurality of molecules. In one still further embodiment, crosslinking the second plurality of molecules includes applying a crosslinking agent to the second plurality of molecules. In another still further embodiment, crosslinking the second plurality of molecules includes applying UV radiation to the second plurality of molecules.

The method may further include blocking molecules wherein each of the blocking molecules includes a head group configured to prevent fouling, a polymer chain, and an anchor group. In one such embodiment, the head group is a zwitterionic group. In one such embodiment, the head group is a polyethylene glycol group. In one such embodiment, the head group is a phosphatidylcholine group. In one such embodiment, the head group is a hydroxyl group. In one such embodiment, the polymer chain is a polyalkane chain. In one such embodiment, the polymer chain is a polyalkene chain. In one such embodiment, the polymer chain is a polyethylene glycol chain. In one such embodiment, the polymer chain is a polypropylene chain. In one such embodiment, the anchor group is a thiol anchor group. In one such embodiment, the anchor group is a silane anchor group. In one such embodiment, the anchor group is a phosphate anchor group.

In one embodiment, the anchor group is bound to a surface selected from the group consisting of the electrode surface and the blocking layer surface and the anchor group is selected based on the surface it is bound to. In a further embodiment, the pair of the surface and the anchor group is a gold electrode surface and a thiol anchor group. In a different further embodiment, the pair of the surface and the anchor group is a silver electrode surface and a thiol anchor group. In yet another different further embodiment, the pair of the surface and the anchor group is a glass electrode surface and a silane anchor group. In a different further embodiment still, the pair of the surface and the anchor group is a silicon electrode surface and a silane anchor group. In another different further embodiment, the pair of the surface and the anchor group is a metal oxide blocking layer surface and a silane anchor group. In another different further embodiment, the pair of the surface and the anchor group is a metal oxide blocking layer surface and a phosphate anchor group.

In another embodiment where the method comprises binding at least one of the antifouling layer or the protective membrane to the aptamer sensor, the protective membrane includes polybetaine. In a further embodiment thereof, the method further includes crosslinking the polybetaine by applying UV radiation. In another further embodiment, the method further includes crosslinking the polybetaine by applying a crosslinking agent.

The method may further include using the aptamer sensor for a time and applying a negative voltage to the electrode during at least a portion of the time to reduce desorption of at least one of the blocking layer or the aptamer. In a further embodiment, the electrode has a negative absolute voltage limit, the method further including applying the negative absolute voltage limit to the electrode for a duration. In an even further embodiment thereof, the duration is at least 100% of the time. In another even further embodiment, the duration is at least 90% of the time. In another even further embodiment, the duration is at least 50% of the time. In another even further embodiment, the duration is at least 20% of the time. In another even further embodiment, the duration is at least 10% of the time. In another even further embodiment, the duration is at least 5% of the time. In another even further embodiment, the duration is at least 1% of the time.

In another embodiment where the method includes using the aptamer sensor for a time and applying a negative voltage to the electrode during at least a portion of the time to reduce desorption of at least one of the blocking layer or the aptamer, the electrode has a negative average voltage, and the method further including applying the negative average voltage to the electrode for a duration. In an even further embodiment thereof, the duration is at least 100% of the time. In another even further embodiment, the duration is at least 90% of the time. In another even further embodiment, the duration is at least 50% of the time. In another even further embodiment, the duration is at least 20% of the time. In another even further embodiment, the duration is at least 10% of the time. In another even further embodiment, the duration is at least 5% of the time. In another even further embodiment, the duration is at least 1% of the time.

### BRIEF DESCRIPTION OF THE DRAWINGS

The objects and advantages of the disclosed invention will be further appreciated in light of the following detailed descriptions and drawings in which:

FIG. 1A is a schematic of a conventional prior art sensor device.

FIG. 1B is a schematic of a conventional prior art sensor device.

FIG. 2 is a graph showing the sensing performance for a hexane-thiol blocking layer.

FIG. 3 is a schematic of a sensor device with a blocking layer having anti-fouling properties.

FIG. 3A is a graph showing the sensing performance for a mercaptohexanol (MCH) blocking layer and for a mercaptooctanol (MCO) blocking layer on rough gold in Serum.

FIG. 3B is a graph showing the sensing performance for a mercaptohexanol (MCH) blocking layer and for a mercaptooctanol (MCO) blocking layer on rough gold in Serum+50 mM ethylenediaminetetraacetic acid (EDTA).

FIG. 3C is a graph showing the sensing performance for a mercaptohexanol (MCH) blocking layer on rough gold in Serum+50 mM ethylenediaminetetraacetic acid (EDTA).

FIG. 3D is a graph showing the sensing performance for a mercaptooctanol (MCO) blocking layer on rough gold in Serum+50 mM ethylenediaminetetraacetic acid (EDTA).

FIG. 4A is a graph showing titration of vancomycin with a mercaptooctanol (MCO) blocking layer at Day 0.

FIG. 4B is a graph showing titration of vancomycin with a mercaptooctanol (MCO) blocking layer at Day 3.

FIG. 5A is a schematic of a sensor device with a blocking layer having anti-fouling properties and a protective membrane layer.

FIG. 5B is a schematic of a sensor device with a blocking layer having anti-fouling properties and a protective membrane layer.

FIG. 5C is a schematic of a sensor device with a blocking layer having anti-fouling properties and a protective membrane layer.

FIG. 5D is a graph showing the performance of a cross-linked polybetaine protective membrane layer for fouling resistance in serum.

FIG. 6 is a schematic of a sensor device with a non-monolayer blocking layer having anti-fouling properties and a protective membrane layer.

### DEFINITIONS

As used herein, “continuous sensing” with a “continuous sensor” means a sensor that changes in response to changing concentration of at least one solute in a solution such as an analyte. Similarly, as used herein, “continuous monitoring” means the capability of a device to provide multiple measurements of an analyte over time.

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “electrode” means any material that is electrically conductive such as gold, platinum, nickel, silicon, conductive liquid infused materials such as ionic liquids, PEDOT:PSS, conductive oxides, carbon, boron-doped diamond, nanotubes or nanowire meshes, or other suitable electrically conducting materials.

As used herein, the term “monolayer blocking layer” means a homogeneous or heterogeneous layer of material or of one or more types of molecules on an electrode which reduce electrochemical background current and/or current due to electrochemical interference, and which may promote proper freedom of movement for the aptamer which is required for creating a measurable response to analyte concentration.

As used herein, “monolayer blocking layer defect size” or “blocking layer defect size” or “blocking layer defect density” or “blocking layer fractional defect area” are defined as used by researchers who investigate the organization and defectivity of self-assembled monolayers of molecules on substrates such as gold. For avoidance of doubt, defect size and defect density are defined using the same measurement principles as taught in: Green, J.-B. D., Clarke, E., Porter, M. D., McDermott, C. A., McDermott, M. T., Zhong, C.-J. and Bergren, A. J. (2022), On the Counter-Intuitive Heterogeneous Electron Transfer Barrier Properties of Alkanethiolate Monolayers on Gold: Smooth versus Rough Surfaces, Electroanalysis.

As used herein, the term “non-monolayer blocking layer” means a homogeneous or heterogeneous layer of material or of one or more types of molecules on an electrode which do not represent a monolayer configuration, and which reduces electrochemical background current and/or current due to electrochemical interference, and which may promote proper freedom of movement for the aptamer which is required for creating a measurable response to analyte concentration. For example, a metal or semiconductor oxide can be a non-monolayer blocking layer, or a thin polymer film may be a non-monolayer blocking layer, because they are comprised of multiple layers of atoms or molecules. A single atomic monolayer of SiO2 for example would be a monolayer, whereas 3 nm of SiO2 is a non-monolayer.

As used herein, the term “antifouling layer” means a homogeneous or heterogeneous layer of material or of one or more types of molecules on a surface which reduces fouling on a surface compared to if such an antifouling layer was not utilized.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, affimers and other forms of affinity-based biosensors. Modifications may include substituting unnatural nucleic acid bases for natural bases within the aptamer sequence, replacing natural sequences with unnatural sequences, or other suitable modifications that improve sensor function, but which behave analogous to traditional aptamers. Two or more aptamers bound together can also be referred to as an aptamer (i.e., not separated in solution). Aptamers can have molecular weights of at least 1 kDa, 10 kDa, or 100 kDa.

As used herein, the term “redox tag” or “redox molecule” means any species such as small or large molecules with a redox active portion that when brought adjacent to an electrode can reversibly transfer at least one electron with the electrode. Redox tag or molecule examples include methylene blue, ferrocene, quinones, or other suitable species that satisfy the definition of a redox tag or molecule. In some cases, a redox tag or molecule is referred to as a redox mediator. Redox tags or molecules may also exchange electrons or change in behavior when brought into proximity with other redox tags or molecules. Exogenous redox molecules are those added to a device, e.g., they are not endogenous and provided by the sample fluid to be tested.

As used herein, the term “change in electron transfer” means a redox molecule whose electron transfer with an electrode has changed in a measurable manner. This change in electron transfer can, for example, originate from availability for electron transfer, distance from an electrode, impedance between the redox molecule and the electrode, diffusion rate to or from an electrode, a shift or increase or decrease in electrochemical activity of the redox molecule, or any other embodiment as taught herein that results in a measurable change in electron transfer between the redox molecule and the electrode.

As used herein, the term “sensing monolayer” means at least a plurality of aptamers on a working electrode, which may also include a plurality of molecules or mixtures of molecules that form a blocking layer and/or an anti-fouling layer.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “continuous sensing” simply means the device records a plurality of readings over time.

As used herein, a “sensing device” or “device” comprises at least one sensor based on at least one aptamer and at least one sample solution. Devices can sense multiple samples and be in multiple configurations such as a device to measure a pin-prick of blood, or a microneedle or indwelling sensor needle to measure interstitial fluid, or a device to measure saliva, tears, sweat, or urine sensor, or a device to measure water pollutants or food processing solutes, or other devices which measure at least one analyte found in a sample solution.

As used herein, a “redox current” or “redox signal” comprises the total redox current between the plurality of redox tags attached to aptamers on the sensor and the electrode for a given sensor, the electrode typically referred to as the working electrode, as measured using techniques such as square voltammetry, chronoamperometry, or other suitable methods.

As used herein, “signal gain” comprises a change in the redox current or signal due to a change in concentration of the analyte as measured using techniques such as square voltammetry, chronoamperometry, or other suitable methods.

As used herein, “background current” comprises the current measured that is not the redox current. Background current can be due to capacitive charging, faradic currents, oxygen reduction currents, other redox active species, etc.

As used herein, a “sensor signal” or “normalized sensor signal” comprises redox current minus the background current which is normalized to the beginning of testing of the sensor (such as t=0 s the sensor is placed into test solution such as serum). For example, if the measurement is a voltammogram from square wave voltammetry then than the sensor signal is the peak height of redox current as measured above the background current if there were no redox tags.

As used herein, a “electron transfer rate” comprises the measured rate or time at which electrons are transferred between the redox tag and the working electrode.

As used herein, a “frequency response” refers to a change in signal measured from the device as a function of measurement frequency, such as the frequency used for a square wave voltammetry measurement. A change in frequency response can also be related to a change in the electron transfer rate.

As used herein, a “zero frequency” or “zero gain frequency” is a measurement frequency where the redox signal does not respond to an increase or decrease in concentration of the analyte. A zero gain frequency can be used to enable two frequency measurement which then permits calibration free operation.

As used herein, “sensor accuracy” is the maximum difference that will exist between the actual value (which must be measured by a primary or good secondary standard) and the indicated value at the output of the sensor. The accuracy can be expressed either as a percentage of full scale or in absolute terms.

As used herein, “test fluid” is interstitial fluid or a suitable proxy for the test fluid such as serum.

As used herein, a “protective membrane” refers to one or more layers or materials which protect a sensor blocking layer from fouling and is permeable to at least electrical charge transfer. A protective membrane may optionally also be selectively permeable to additional components in a test fluid such as, for example, at least one analyte, wherein the presence of the at least one analyte allows the sensor to operate properly while the protective membrane protects against performance reduction due to fouling, or some combination thereof.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a reference or counter electrode, a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

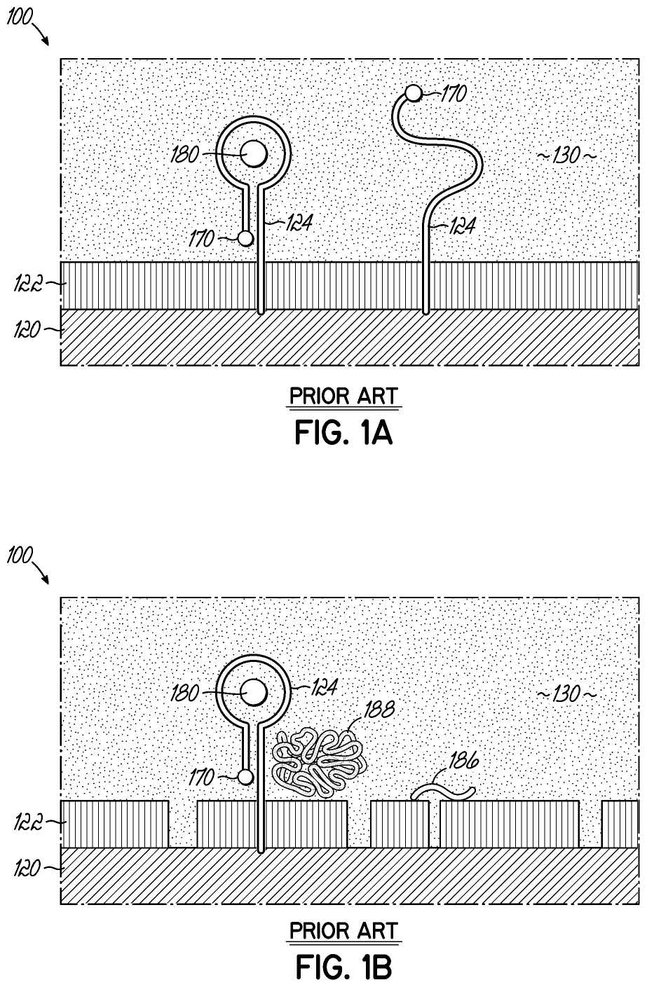

With reference to FIGS. 1A and 1B, a conventional prior art sensor device 100 is shown placed in a sample fluid 130 such as interstitial fluid, comprising: at least one working electrode 120 such as gold, carbon, or other suitable electrode material; at least one monolayer blocking layer 122 including a plurality of molecules such as, for example, mercaptohexanol or hexanethiol that are thiol bonded to the electrode, or a plurality of natural solutes in blood such as, for example, amino acids, peptides, albumin, etc. that can at least partially act as a blocking layer, or other suitable molecules depending on application and on the choice of electrode 120 material; at least one aptamer 124 that is responsive to binding to an analyte 180 and which contains a redox tag 170 such as, for example, methylene blue. In the generic example taught for FIGS. 1A and 1B, the aptamer 124 is a simple stem loop (hairpin) aptamer where analyte 180 binding causes the stem loop to form and the redox current measured from the redox tag 170 to increase, as measured using square wave voltammetry, chronoamperometry, or some other suitable technique. In absence of analyte 180 binding to the aptamer 124 the stem loop conformation does not form and the redox current thus does not increase. Thus, changes in a measurement of redox current can be used as a signal to interpret changes in concentration of the analyte 180.

With further reference to FIG. 1B, a challenge with aptamer sensors is that when placed into initial operation the sample fluid 130, over a period of tens of minutes to hours, the signal (e.g., redox current) initially decreases by 30%, 50%, or even more, due to effects such as fouling by small molecules 186, proteins 188, or other solutes in the sample fluid 130, but also due to desorption of the sensing monolayer, including aptamer 124 and/or blocking layer 122. Various aspects of the present invention aim to reduce or resolve entirely the amount of initial or longer term desorption, instability, and/or fouling of the sensing monolayer. During fabrication of a conventional aptamer sensor, a portion of the sensing monolayer is physiosorbed to the electrode 120 surface, or molecules are incorrectly oriented (e.g., blocking layer that is inverted with thiol facing away from electrode 120), or there are portions of the electrode 120 that are physically or electrochemically fragile, or other aspects of the sensing monolayer that can readily detach from the sensor during initial operation. Although one could just use an electronic ‘burn-in’ period to stabilize the sensor when in initial use, the loss of aptamer 124 results in an irrecoverable loss in redox current from the sensor 100, and the loss of blocking layer 122 can result in an increase in background current and/or increased electrode 120 fouling. Furthermore, even if the sensing monolayer stays intact, if it exhibits significant fouling in a biofluid such as interstitial fluid then the redox current can decrease due to reduced electron transfer from the redox tag 170 or reduced freedom of movement for the aptamer 124 which can also reduce signal gain with binding of the analyte 180. Nuclease attack or methylation of the aptamer 124 are also possible and will degrade the signal from the sensor 120. The measurement of the sensor 100 can be performed using one of multiple techniques, such as chronoamperometry, square-wave voltammetry, using one or more calibration-free and/or drift correction methods, all of which are sensitive to degradation of the sensor 100. In summary, a non-limiting set of challenges may include the following, which have not been fully resolved by researchers in the field of aptamer sensors:

(1) Desorption of the aptamer from the sensing monolayer which reduces the redox current, and therefore sensor accuracy. Energetically stable aptamer attachment to the working electrode is preferred and is measurable by a stable sensor signal. When a sensing monolayer is used, stable aptamer attachment can have a strong dependence on the stability of the monolayer blocking layer that surrounds the aptamer.

(2) Desorption of blocking molecules from the sensing monolayer which increases background current due to increased electrical capacitance or interferents such as oxygen reduction current, and which changes electron-transfer rates between electrode and redox tags thereby also impacting redox current, signal gain, frequency response, and therefore sensor accuracy. Energetically stable blocking layer attachment to the working electrode is preferred.

(3) Nuclease attack, oxidation, or methylation or other chemical attack of the aptamer which can sever the aptamer and release the redox tag and/or inhibit the response of the aptamer when it binds to analyte, all of which is resolvable or at least reducible by modifying the aptamer to be resistant to such attack.

(4) Fouling of the aptamer due to irreversible or frequent binding of a solute to the aptamer that is not the target analyte, which could alter or completely inhibit the aptamer response to the analyte.

(5) Fouling of the blocking layer which can alter the redox current, signal gain, frequency response, and/or rate of electron transfer by changing the electrical impedance between the redox tag and the electrode, and which can inhibit normal motion/response of the aptamer to binding of analyte to the aptamer, which further impacts signal gain and frequency response. Fouling can also accelerate desorption of the blocking molecules in the blocking layer by creating intermediate energy states that reduce the energy required for desorption of blocking molecules. Reduced fouling of the blocking layer is preferred.

(6) Fatigue of the redox tag where its reduction/oxidation becomes no longer reversible, shifted in potential, or some other change in the redox tag.

Of this list, changes in the aptamer (3), fouling of the aptamer (4), and redox tag fatigue (6) can be adequately insignificant during multi-day operation of a sensor when using currently available aptamers and redox tags such, for example, as methylene blue. For a long-lasting sensor that maintains sensor accuracy for multiple days such as, for example, at least 3, 4, or 5 days, the remaining list—(1), (2), and (5)—must be addressed in part and ideally in whole, as will be further taught for embodiments of the present invention. The present invention will therefore be organized into examples and embodiments that provide one or more of: (1) a stable sensor signal; (2) an energetically stable blocking layer; (5) resistance to fouling of the blocking layer.

Several examples and embodiments of the present invention will now be taught, and their performance then subsequently summarized. As will be seen, several of the examples are surprising, revealing that previously-known short term fixes to improving sensor longevity (over hours) may actually increase sensing monolayer degradation in the longer term (over days). For example, it was previous thought that solutes in serum would stabilize a sensing monolayer, which may be true in the short term (hours) but is not true over periods of 3 days or more, requiring an alternate strategy for creating a stable sensor (for example, see “Achieving Reproducible Performance of Electrochemical, Folding Aptamer-Based Sensors on Microelectrodes: Challenges and Prospects” Anal. Chem. 2014, 86, 22, 11417-11424, Oct. 22, 2014, https://doi.org/10.1021/ac503407e). Furthermore, it has been observed that electric field during scanning of the sensors can energetically drive off the molecules in the sensing monolayer and therefore less frequent measurement may improve sensor longevity, which may be true in the short term (hours) but may not be true over periods of 3 days or more, where applied electric field or voltage can actually help further stabilize the sensing monolayer.

Unless specifically stated otherwise, examples using an aptamer utilized the vancomycin aptamer that is commonly reported in literature having the following formulation: CGAGG GTACC GCAAT AGTAC TTATT GTTCG CCTAT TGTGG GTCGG, with a carbon thiol linker on the 5′ end and methylene blue on the 3′ end. This aptamer was chosen for reproduction of results. Embodiments of the invention may include other aptamers known to persons having ordinary skill in the art than those aptamers disclosed here, either as a substitution for or in addition to the vancomycin aptamer disclosed above.

For sensor fabrication, 2 mm diameter gold disk electrodes were used and mechanically roughened via abrasion or physical agitation in polishing slurries. The working electrode was further cleaned by running 700 cyclo-voltametric scans in 0.5 M NaOH from −1 V to −1.6 V at a scan rate of 1 V/s and subsequently 150 scans in 0.5 M H₂SO₄ solution by scanning the potential from 0 V to 1.6 V at 1 V/s. Once the electrochemical cleaning was completed, electrodes were thoroughly rinsed with DI water, dried in the nitrogen stream (99.999% purity) and used for the preparation of the sensors. Embodiments of the invention may include other types of electrodes such as, for example, gold wire, planar deposited gold, another suitable gold working electrode, and/or other types of working, counter, or reference electrode formulations, so long as they satisfy the sensor properties and performance as taught herein. Embodiments of the invention may also include electrodes having larger or smaller dimensions.

Unless specifically stated otherwise, the aptamers were bound to the electrodes as follows. First, a 100 μM vancomycin aptamer stock solution was prepared in TE buffer, including both tris(hydroxymethyl)aminomethane (Tris) and ethylenediaminetetraacetic acid (EDTA), and kept at −20° C. until used. The preparation of the aptamer solution for aptamer incubation onto the electrodes was performed by mixing an aliquot of the 100 μM vancomycin stock solution with equal volume of the 0.5 M TCEP (tris(2-carboxyethyl) phosphine). The mixture was allowed to rest for 1 h, until the reduction of the thiolated aptamer was completed. The obtained solution was diluted to 500 nM with 1× phosphene buffered saline (PBS) with addition of 2 mM MgCl₂ and used for the functionalization of the gold-disk electrodes. A 20 μL droplet of the so-obtained reduced aptamer solution was drop casted over the electrochemically cleaned gold working electrode surface and left to incubate for 1 h in a light-protected and humidity-controlled chamber. The aptamer functionalized electrodes were rinsed with DI water and incubated for at least 12 hours in 5 mM solution of blocking layer molecules prepared in 1×PBS (buffer solution). The functionalized sensors were rinsed with DI water prior to measurement. In all cases, purity of solutions used during deposition are critical and otherwise will compromise the sensor results, the purity not being just the purchased purity but ensuring that all lab-ware and solutions used are clean and free from significant impurities that will compromise formation of a stable sensing monolayer. Generally, purity of solutions may have been discounted in prior work because prior work did not understand the mechanistic degradation mechanisms that result in desorption of the sensing monolayer.

All electrochemical measurements were performed by CH Instruments galvanostat/potentiostat (Austin, Texas) connected to 64 channel multiplexers in a standard three-electrode system with the gold disk electrode serving as working electrode, platinum counter electrode and Ag/AgCl (3 M KCl) reference electrodes tested under temperature and humidity-controlled conditions. Cyclic voltammograms were recorded in a potential window from −0.1 V to −0.5 V at a scan rate of 100 mV/s. For determining the electron-transfer rate constant a set of cyclic voltammograms was recorded at different scan rates ranging from 5 mV/s to 100 V/s. Square-wave voltammetry was performed in a potential window from −0.1 V to −0.5 V at 25 mV amplitude and unless otherwise specifically noted using a 300 Hz scanning frequency. For the longevity experiments, serum was spiked with sodium-azide to a final concentration of 0.02% wt. to prevent growth of microorganisms. Averaged sensor measurements were performed across 4 sensors for each sensor type, and in comparative tests there were 4 sensors and 2 types for 8 total electrodes such that cycling through each electrode occurred approximately every 30 s, during which each electrode experienced voltage for approximately 2 s to complete the scan on each electrode.

### EXAMPLES

### Example 1: Biosensor Including a Stable Sensor Signal, and an Energetically Stable Blocking Layer, but No Fouling Resistance

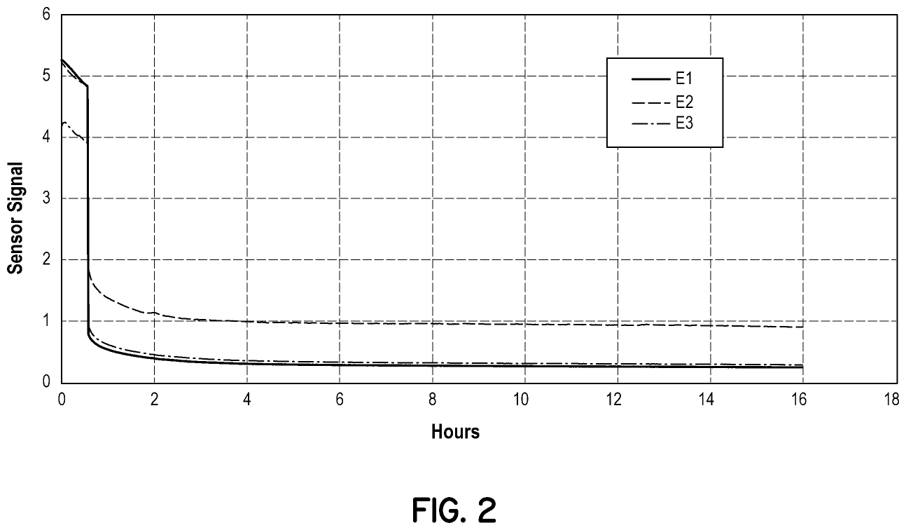

With reference to FIG. 2, an example of a conventional sensor using a hexane-thiol blocking layer is shown to be inadequate at least because it experiences sensor signal degradation over time. Hexane-thiol provides stability in background current because the hexane-thiol is energetically more stable on the gold working electrode, but suffers from significant fouling of the blocking layer due to the hydrophobicity at the terminus of the hexane-thiol molecules (the terminus end faces the solution or sample fluid). The abrupt drop in sensor signal for electrode 1 (E1), electrode 2 (E2), and electrode 3 (E3) occurs when the sensors are placed into a sample fluid of serum. The sensor signal decrease rapidly occurs over 1-2 hours and is approximately an 80% loss of signal due to rapid fouling of the blocking layer in serum which decreases the redox current. Common foulants include albumin, which has hydrophobicity which easily adheres to the hydrophobicity of hexane-thiol blocking layer. The sensors also rapidly decrease in signal gain (not shown), to a level of poor performance, because the fouling is so strong that a build up of proteins like albumin will actually restrict freedom of movement of the aptamers. Therefore, while providing energetic stability for the blocking layer, this example is not a viable solution for a sensor with long-lasting signal gain and sensor accuracy. The strong fouling of the sensors in FIG. 2 occurred across aptamers tested for analytes such as phenylalanine, vancomycin, and cortisol, confirming that the reduction in sensor performance is due to fouling of the blocking layer.

The following examples describe and build on aspects of the present invention. Aspects of the present invention may be used together or in isolation (for example and antifouling blocking layer and/or a protective hydrogel against fouling). Therefore, each example need not explicitly teach all such uses of aspects of the present invention when used together or in isolation.

### Example 2: Biosensor Including Stable Sensor Signal, Energetically Stable Blocking Layer, and Moderate Fouling Resistance

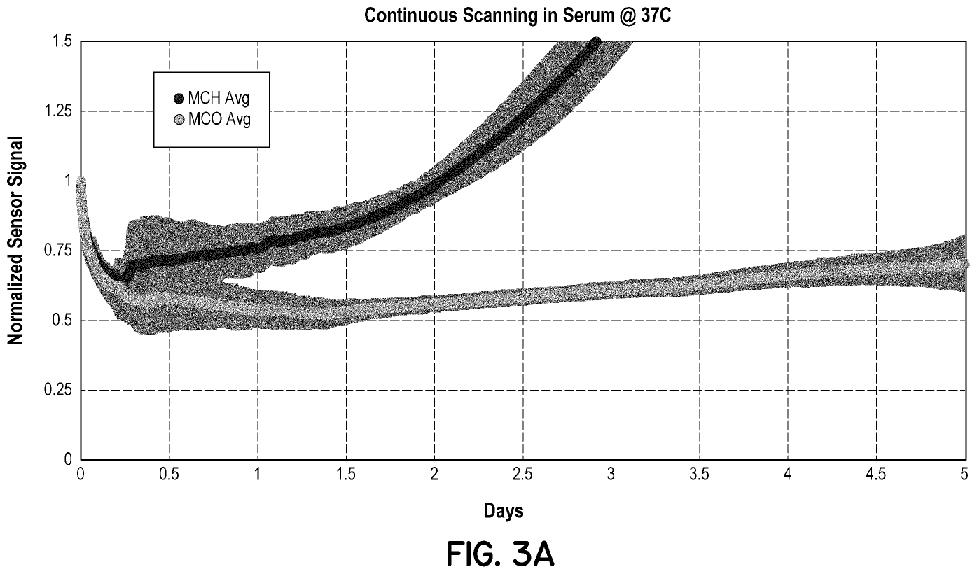

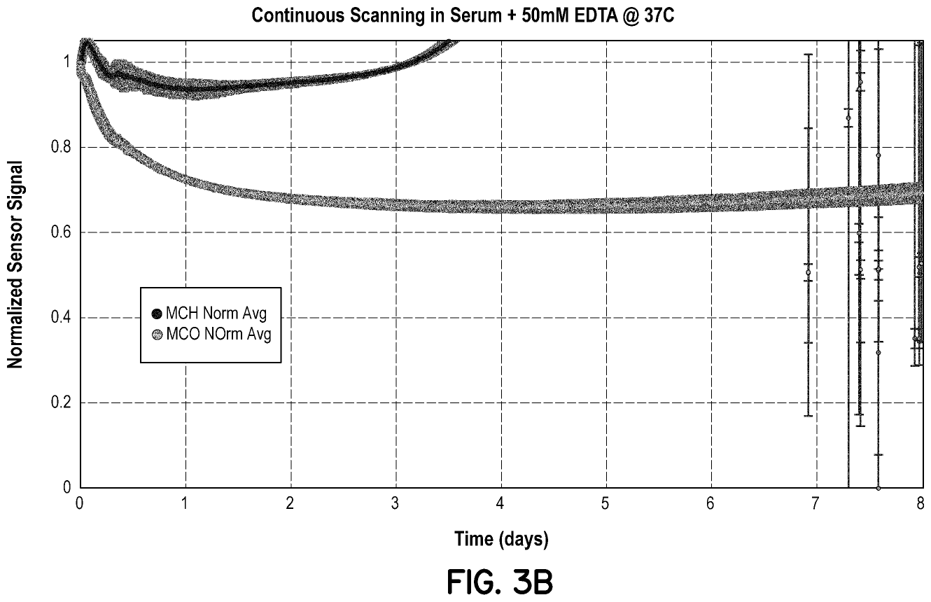

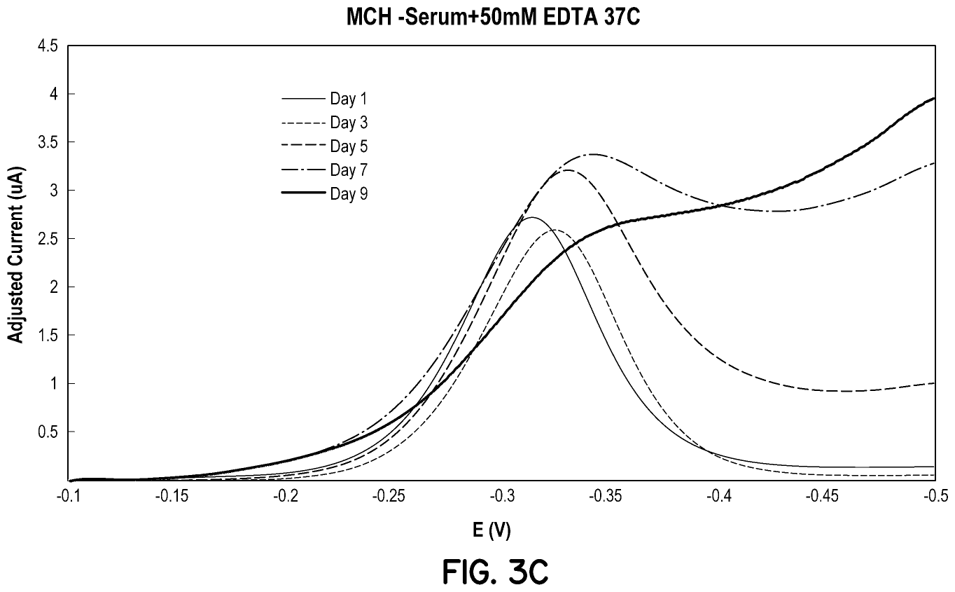

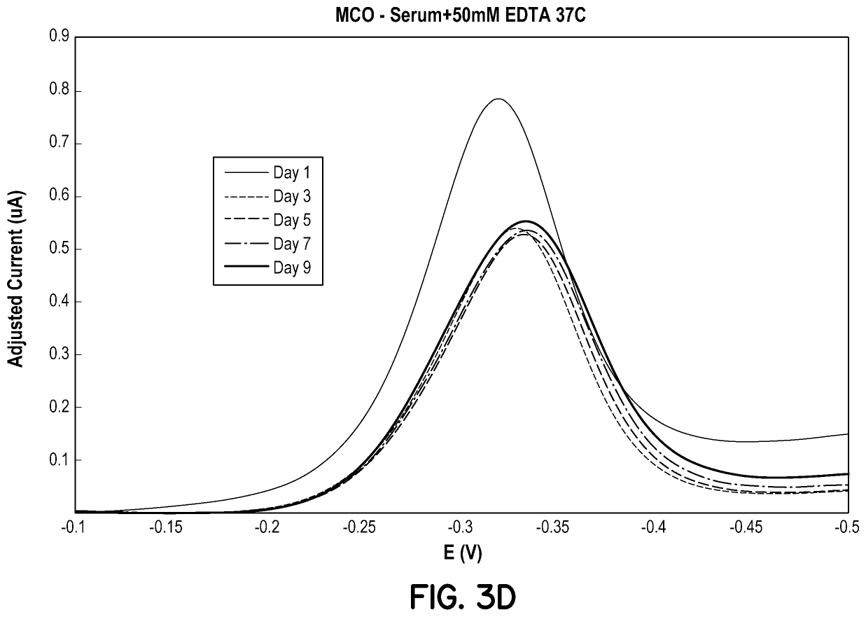

With reference to FIG. 3, where like numerals refer to like components of FIG. 1A unless specified otherwise, an embodiment of a sensor device 300 in accordance with the principles of the present invention is shown. The sensor device 300 includes at least one working electrode 320, at least one blocking layer 322, at least one aptamer 324 that is responsive to binding to an analyte 380 and which contains a redox tag 370. In one embodiment, the at least one working electrode 320 includes a material selected from the group consisting of gold, carbon, or another suitable electrode material. In one embodiment, the at least one aptamer 324 is the vancomycin aptamer which, like other possible aptamers, can be used to implement electrochemical measurement techniques such as, for example, square wave voltammetry, chronoamperometry, or some other suitable technique. In such an embodiment, the absence of the analyte 380 binding to the aptamer 324 results in in a decrease in redox current. The sensor device 300 differs from the conventional sensor device 100 at least because of the differences between the composition of the blocking layer 122 and the composition of the blocking layer 322. In one embodiment, the blocking layer 322 imparts a fouling resistance to the sensor device 300. In another embodiment, the blocking layer 322 is stable for greater than or equal to 3 days, more preferably stable for greater than or equal to 5 days at a temperature greater than or equal to 30° C. In a further embodiment, the blocking layer 322 is stable for greater than or equal to 3 days, more preferably stable for greater than or equal to 5 days at a temperature greater than or equal to 30° C. and less than or equal to 47° C. In a further embodiment, the blocking layer 322 includes a plurality of molecules that impart a fouling resistance and longevity to the sensor device 300 such as, for example, mercaptooctanol (MCO) or other suitable alkylthiols or blocking molecules having a binding energy on rough gold that inhibits significant desorption of the blocking layer for at least 3 days at a temperature greater than or equal to 30° C. In a further embodiment, the blocking layer 322 includes a plurality of molecules that impart fouling resistance and longevity to the sensor device 300 such as, for example, MCO or other suitable alkylthiols or blocking molecules having a binding energy on rough gold that inhibits significant desorption of the blocking layer at a temperature greater than or equal to 30° C. and less than or equal to 47° C.

With reference to FIGS. 3A, 3B, and embodiments of the present invention, data is shown for a vancomycin sensing device that has a stable sensor signal, energetically stable blocking layer, and moderate fouling resistance. The results for a mercaptooctanol (MCO) blocking layer are shown on mechanically polished gold disks of 2 mm diameter and compared to a much less stable mercaptohexanol (MCH) blocking layer fabricated in the same manner as the MCO. Both the sensors and blocking layers of MCH and MCO were fabricated identically except for the chemical composition associated with MCH vs. MCO. With further reference to FIG. 3A, 3B, the sensors are repeatedly scanned with square wave voltammetry during data collection. The sensors are tested in serum at 37° C. in order to test against stable sensor signal, an energetically stable blocking layer, and resistance to fouling.

The plots measure sensor signal vs. time for multiple sensors and include standard deviation for the sensor signal (gray shading outside the sensor colored raw redox current data). The MCO sensor exhibits superior longevity as indicated by greater than or equal to 50% of sensor signal remaining for a period of time greater than or equal to 3, 4, or 5 days of operation at a temperature of 37° C., which is significantly greater than a typical lower limit for a dermal indwelling sensor of greater than or equal to 30° C. The MCH layer sensor signal is less stable as indicated by the measured increase in signal, which is evidence of sensor degradation as will be discussed in more detail below with regard to FIG. 3C. The MCO layer still has an initial fouling response resulting in 50% sensor signal loss which is similar to that shown for MCH as well, but which is superior to the ˜80% sensor signal loss due to fouling of the hexanethiol monolayer blocking layer of FIG. 2. The MCO blocking layer is energetically more stable due to a greater energy needed for desorption from the monolayer, achieved by a longer hydrophobic chain (8 vs. 6 carbons) compared to commonly used MCH blocking layers. The thiol-gold bond can be described as a surface-bound thiolate. The gold-thiolate bond energy is approximately only 170 KJ/mol due to the polar nature of the bond, causing it to be regarded as a pseudo-covalent bond. Van der Waals forces between neighboring molecules stabilize the structure further with a greater total binding energy. For alkythiols on 111 oriented gold, the total binding energy of MCH is-2.98 eV whereas the total binding energy of MCO is −3.17. This small binding energy increase for MCO is significant, as predicted by the Arrhenius equation, k=Ae\hat{ }(−E/RT), where E is the binding energy. The binding energy difference between −2.98 eV and −3.17 eV is significant enough to raise a maximum stable temperature of the sensor by nearly 20° C. (or Kelvin), which explains both why MCH can be shown to be stable for several days at room temperature but is instable at greater than or equal to 30° C. and why MCO is highly stable even at temperatures greater than or equal to 30° C. These total binding energy stabilities are further dependent on an underlying gold surface with optimal roughness and crystallinity, as will be taught later as part of embodiments of the present invention. In one embodiment, molecules in the blocking monolayer of the present invention may include those having a total binding energy of that is equal to or more negative than −3.00 eV, more preferably still equal to or more negative than −3.05 eV, and even more preferably equal to or more negative than −3.1 eV.

With further reference to FIG. 3B, 50 mM Ethylenediaminetetraacetic acid (EDTA) was added to confirm that nuclease attack was not causing the degradation. In FIGS. 3B, 3C and 3D the signal and the voltammograms for MCH and MCO respectively are shown at 1, 3, 5, 7, and 9 days. These graphs confirm, that even in the presence of 50 mM EDTA, the MCH blocking layer is unstable past 3 days as increased background accelerates after 3 days due to both increased capacitive charging and increased oxygen reduction current (which occurs at larger negative voltages). The MCO electrode is stable for greater than one week even at 37° C. and in serum. Referring back to FIG. 3A, which is without added EDTA and therefore more representative of a natural biofluid such as interstitial fluid, the device with the MCH blocking layer begins to fail after only 36 hours, which is far short of 3 days, while the MCO blocking layer remains stable for at least 3, 4, or 5 days.

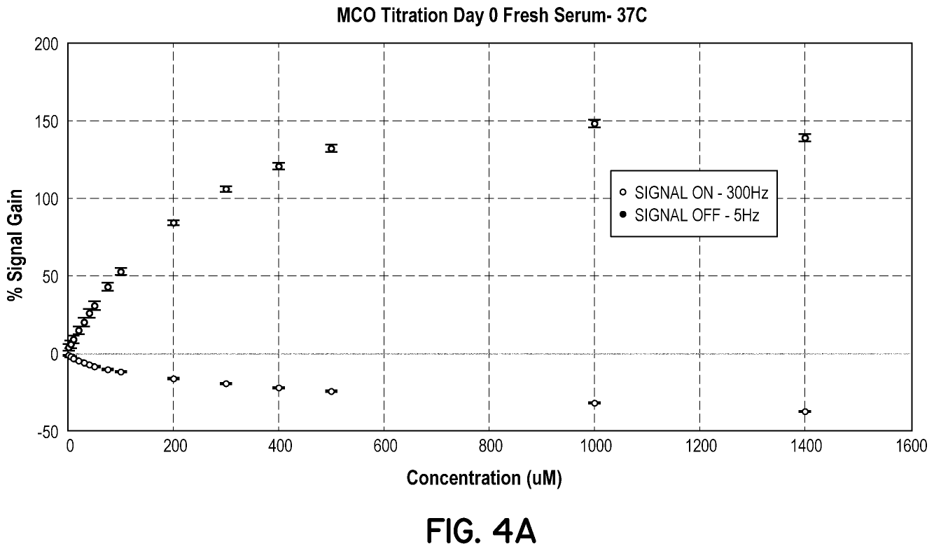

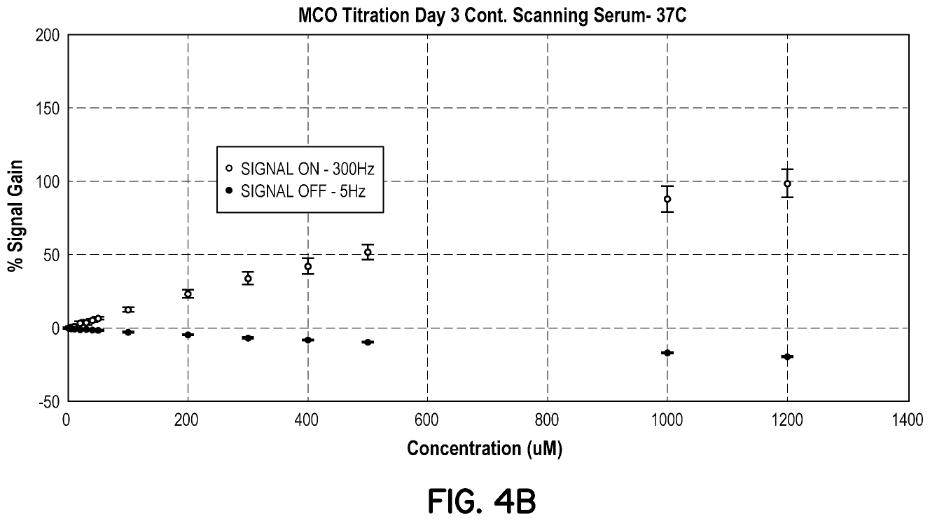

The results in FIGS. 3A-3D only partially resolve all the ideal performance metrics for a long-lasting stable sensor because sensor signal is not the only metric that matters. For example, as shown in FIGS. 4A and 4B, the signal gain in response to vancomycin decreases significantly after 3 days for the same sensors due to partial fouling of the MCO blocking layer surface. For example, after 3 days of continuous scanning in a 200 μM MCO solution at 37° C., the signal gain of the sensor decreased from about 80% to about 25% (approximately 4 times), a decrease of less than four times of its original value (i.e., preserves greater than one fourth of its original signal gain value). This 25% signal gain still permits an accurate reading using two-frequency calibration free measurements but has reduced accuracy due to weaker signal gain and a shift in the sensor frequency response. This continued loss in signal gain and shift in frequency response is due to fouling of the MCO layer which inhibits or alters electron transfer and/or freedom of movement for the aptamer. Both fouling and monolayer passivation have a strong dependency on feature types and densities of the monolayer, which will be discussed next for embodiments of the present invention.

### Example 3: Electrode Roughness and Monolayer Blocking Layer Defects for Stable Sensor Signal, Energetically Stable Blocking Layer, and Fouling Resistance

With reference to embodiments of the present invention, not only does the choice of blocking layer effect sensor signal stability and fouling resistance, but sensor signal stability and fouling resistance can both be strongly affected by the underlying electrode crystallinity and roughness. For example, a perfectly planar or highly crystalline gold electrode can actually bias the size distribution of features in the blocking monolayer toward features that are larger in size, resulting in a less energetically stable blocking layer than a blocking layer formed on rougher gold, which has smaller sized and more energetically stable defects due, at least in part, to the step edges in rougher gold. Furthermore, the right density of features can be beneficial as features promote electron transfer between the redox tag and the working electrode as explained further below in the discussion of defects. Features can include, for example, defects in the blocking layer, electrically conductive molecules, pores, other suitable means of promoting electron transfer, or combinations thereof. With respect to defects, larger defects can also act as sites where undesirable fouling nucleates more quickly. Therefore, an optimum surface density and size of monolayer defects is desired, all of which is affected by electrode crystallinity and roughness. For example, to ensure that an adequate density of blocking layer defects occur at step edges of a gold surface, the gold may have a roughness that has at least 1's or 10's of nm changes in height of the gold over distances of 10's to 100's of nm in the perpendicular plane (i.e., the width). This roughness of the gold can be expressed using average roughness slope, which is a ratio between the change in step height over a given width. For example, gold that has a 1 nm change in step height (tangential to the local surface or radii of curvature) over 10 nm of width of planar gold domain has a 10% average roughness slope to it. Therefore, an embodiment of the present invention may utilize gold with an average slope roughness of less than or equal to at least one of 0.5%, 1%, 2%, 5%, 10%, 20%, or 50%. Roughness of the electrode may be at least partially dependent on the stability of the electrode material. For example, a conventional electrochemically roughened gold electrode may exhibit mechanically, thermally, and chemically unstable features such as nano-scale gold dendrites, nano-porous structures, other unstable features, or some combination thereof. For example, nano-porous gold has a roughness and porosity that can change over time (decrease) even at room or body temperature as the gold self-anneals and changes in geometry over time. In one embodiment, the gold electrode may have an average slope roughness of less than or equal to at least one of 0.5%, 1%, 2%, 5%, 10%, 20%, or 50% after annealing of unstable features. In another embodiment, the gold electrode may have an average slope roughness of less than or equal to at least one of 0.5%, 1%, 2%, 5%, 10%, 20%, or 50% before annealing of unstable features.

With further reference to embodiments of the present invention, a perfectly defect-free blocking layer with little or no tunneling current could make for a poor biosensor because, for example, electron transfer with the redox-tag could be inhibited and/or electron transfer kinetics could be too slow and therefore limit the availability of a zero gain frequency. Conversely, a too highly defective blocking layer would have too much background current from capacitive charging from molecular interferents in the sample fluid and/or due to oxygen reduction current. Therefore, a balance or ‘goldilocks’ zone for the right density and size of monolayer blocking layer defects is preferred. The size and density of defects in the blocking layer can be determined using a measurement technique such as, for example, an optical method, AFM, scanning tunneling microscopy, or other suitable measurement techniques. In a preferred embodiment, the majority of blocking layer defect sizes may be less than or equal to 0.3 nm in size. In various embodiments, the blocking layer has defects with a fractional area of the total surface area that is, in various embodiments, less than 0.2, less than 0.1, less than 0.05, or even less than 0.02. In various embodiments, layer 122 has defects that have a fractional area of the total surface area that is, in various embodiments, at least greater than 0.001, greater than 0.002, greater than 0.005, greater than 0.01. The defect size is typically measured as a width of a defect between adjacent ordered domains of the blocking layer molecules and in an image of the scan appears as a void or crack in between adjacent ordered domains of the blocking layer molecules. A smaller defect size promotes energetic stability of the monolayer at least because blocking layer molecules that have a hydrophobic interior region, such as alkythiols, experience a local energy minima when they are tightly packed (i.e., surrounded) with similar blocking layer molecules (i.e., it is more difficult to desorb a blocking layer molecule from such a layer). In one embodiment, the blocking layer has a fractional defect area, which is a measure of the area of the underlying electrode that is exposed by the defects as a fraction of total electrode area. In some embodiments, the fractional defect area may be at least 0.01 for the majority of the defect sizes. The blocking layer defect sizes and densities can be controlled by the roughness and crystallinity of the electrode. As an example, two extremes of such roughness and crystallinity control can be achieved for example by sputtering or evaporating an electrode such as gold a rate of greater than or equal to 10 nm/min to create very rough electrodes with a high density of small defects (less than or equal to 0.3 nm), to instead annealing of gold electrodes at 400-500° C. for several hours to smoothen and crystalize the electrode surface and result in a high density of large defects such as, for example, greater than or equal to 0.3 nm.

### Example 4: Stable Sensor Signal, Energetically Stable Blocking Layer, and Strong Fouling Resistance Via Addition of a Protective Membrane

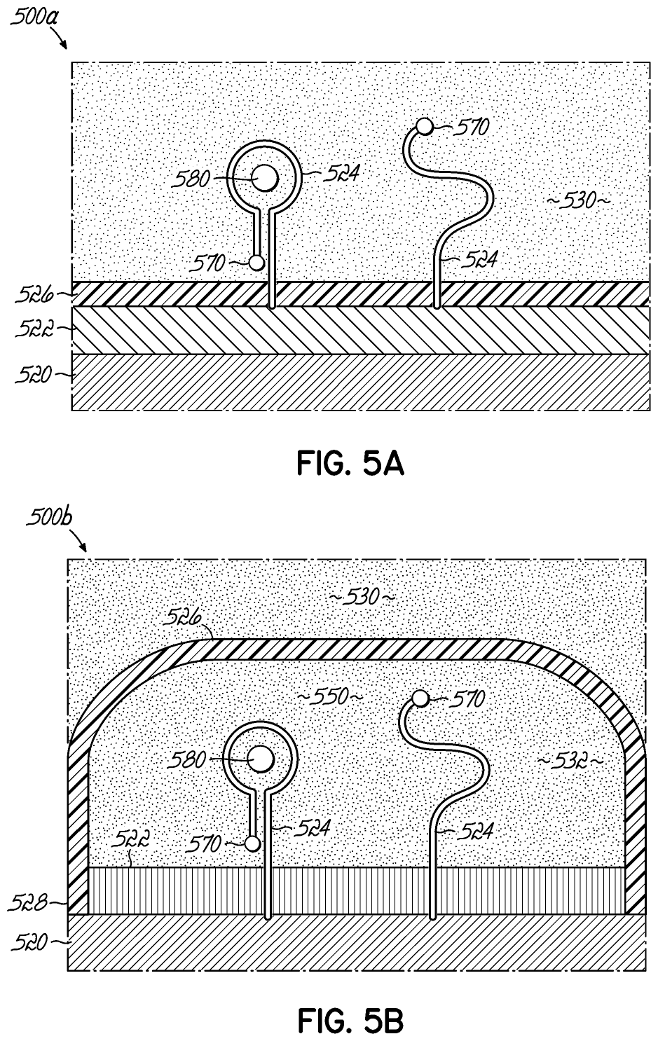

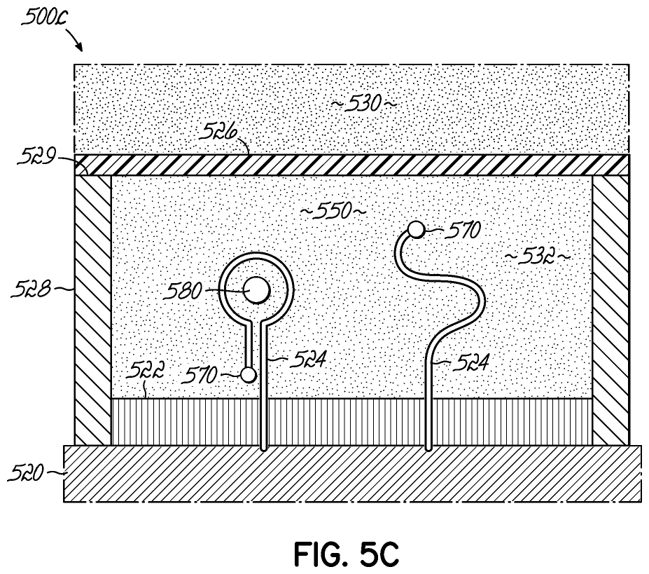

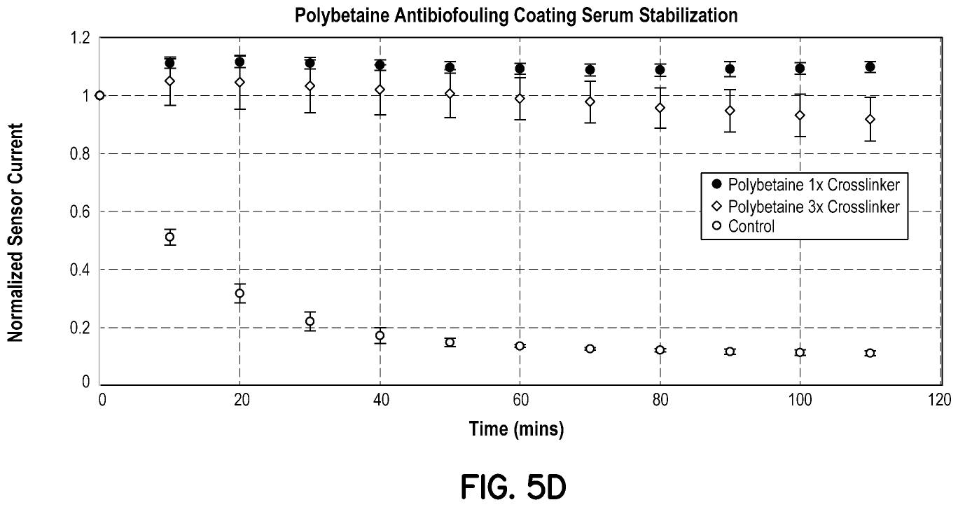

With reference to FIGS. 5A-5C, where like numerals refer to like features shown and described in FIGS. 1 and 3, a protective membrane layer 526 may be added to the device 500a, 500b, or 500c. With reference to FIG. 5A, the device 500a includes the protective membrane layer 526 may be positioned above the blocking layer 522. In one embodiment, the protective membrane layer 526 may be positioned such that a majority of the protective membrane layer 526 covers and is in contact with a majority of blocking layer 522. In such embodiments, if the protective membrane is so dense that it can block free motion of the aptamer 524, then the thickness of the protective membrane layer 526 may be inversely correlated with electron transfer of the device 500a and the membrane 526 may be less than or equal to 10 nm, less than or equal to 5 nm, and even more preferably less than or equal to 1 nm, such as being an ultrathin antifouling layer of a glycol or monolayer of a zwitter ion moiety. If the protective membrane 526 does not block free motion of the aptamer 524 such as being a semiporous hydrogel in which the aptamer can freely move, then the thickness of the protective membrane 526 may be less than or equal to 1 mm, more preferably less than or equal to 100 microns, and even more preferably less than or equal to 10 microns. The protective membrane may be for example a hydrogel such as a solution of betaine that is solution cast onto to the blocking layer 522 and then crosslinked chemically or with ultra-violet light to form a polybetaine protective hydrogel.

With reference to FIG. 5B, a device 500b in accordance with principles of the present invention is shown. As shown, the protective membrane layer 526 may be attached at or around an outer perimeter 528 as shown. Although the outer perimeter 528 is shown to be at or near an edge of the electrode 520, the outer perimeter 528 may encompass the electrode 520 without contacting the electrode 520 (not shown). In one embodiment, the protective membrane layer 526 forms an enclosed space 550 above the blocking layer 522 and encompasses the blocking layer 522 such that any sample fluid such as a test fluid 530 comes in contact with the protective membrane layer 526 before the aptamer 524. In one embodiment, the test fluid 530 is filtered through the protective membrane layer 526 as it enters the enclosed space 550, removing at least one solute present in the test fluid 530, to define a filtered test fluid 532 as shown. The at least one solute filtered out by the protective membrane 526 to define the filtered test fluid 532 may be, for example, albumin, while the analyte such as vancomycin may freely exchange between test fluid 530 and filtered test fluid 532. In one embodiment, the enclosed space 550 is sized so that the at least one aptamer 524 is separated from contact with the protective membrane layer 526. In one embodiment, at least one of the height of the protective membrane layer 526 and the maximum height of the protective membrane 526 is selected to minimize contact between the aptamer 524 and the protective membrane 526.

With reference to FIG. 5C, a device 500c in accordance with principles of the present invention is shown. As shown, the outer perimeter 528 is a raised perimeter 528 having a top surface 529 on which the protective membrane layer 526 is applied. Although the raised perimeter 528 is shown to be at or near an edge of the electrode 520, the raised perimeter 528 may encompass the electrode 520 without contacting the electrode 520 (not shown). In one embodiment, the test fluid 530 is filtered through the protective membrane layer 526 as it enters the enclosed space 550, removing at least one solute present in the test fluid 530, to define a filtered test fluid 532 as shown. The at least one solute filtered out by the protective membrane 526 to define the filtered test fluid 532 may be, for example, albumin, while the analyte such as vancomycin may freely exchange between test fluid 530 and filtered test fluid 532. The raised perimeter 528 may be impermeable to fluids such as, for example, the test fluid 530, the interstitial fluid, or serum. In one embodiment, the raised perimeter 528 and the protective membrane layer 526 form an enclosed space 550 above the blocking layer 522. In one embodiment, the raised perimeter 528 may have a height between the top portion 529 and the blocking layer 522 greater than the height of the aptamer 524 such that the protective membrane layer 526 is separated from the aptamer 524. In one embodiment, the enclosed space 550 is sized so that the at least one aptamer 524 is separated from contact with the protective membrane layer 526.

With reference to FIGS. 5B and 5C, the perimeter 528 may have a cross section perpendicular to the height that defines a perimeter shape. The perimeter shape may be polygonal such as, for example, a triangle, a square, a rectangle, a rhombus, a hexagon, an octagon, or other suitable polygonal shapes. The perimeter shape may have one or more rounded edges such as, for example, a circle, a semi-circle, or a modified polygon with curved edges. Similarly, the electrode 520 may have a cross section perpendicular to the height that defines an electrode shape. The electrode shape may be polygonal such as, for example, a triangle, a square, a rectangle, a rhombus, a hexagon, an octagon, or other suitable polygonal shapes. The electrode shape may have one or more rounded edges such as, for example, a circle, a semi-circle, or a modified polygon with curved edges. In one embodiment, the perimeter shape is selected to match the electrode shape. With further reference to embodiments of the present invention alternate membrane geometries are possible. For example, if a gold-wire working electrode was utilized, the protective membrane may be a dialysis tube into which the gold wire is inserted (not shown).

With reference to FIGS. 5A-5C, the protective membrane layer 526 may be adhered to the blocking layer 522 and/or the perimeter 528 by attachment methods such as, for example, chemically bonding the protective membrane layer 526 to the blocking layer 522 and/or the perimeter 528, mechanically adhering the protective membrane layer 526 to the blocking layer 522 and/or the perimeter 528, applying an adhesive at least at a point of contact between the protective membrane layer 526 and the blocking layer 522 and/or the perimeter 528, some other suitable methods of adhering two surfaces together, or some combination thereof. In one embodiment, the protective membrane layer 526 is partially protective against fouling. A protective membrane layer 526 may include a polymer material such as, for example, polybetaine, polyurethane, polyvinylpyridin, polyvinylimidazole, or some other suitable polymer. In a further embodiment, the protective membrane layer 526 is crosslinked using a method such as, but not limited to, using a crosslinking agent, applying UV radiation, using another suitable cross-linking method, or some combination thereof. In one embodiment, the protective membrane layer 526 is a thin film that is applied on top of a device 500. In a further embodiment, the thin film protective membrane layer 526 has a thickness less than or equal to 1 mm, more preferably less than or equal to 100 microns, and even more preferably less than or equal to 10 microns. In another example, polybetaine may be used in a protective membrane layer 526 that can be coated onto the blocking layer 522 as a solution, and cross-linked with UV light such that is forms a protective membrane layer 526 that is adhered chemically, mechanically, or through other forces at least in part to the surface formed by blocking layer 522 and aptamers 524.

With reference to FIG. 5D for a vancomycin sensing device and with reference to embodiments of the present invention, a stable sensor signal, energetically stable blocking layer, and fouling resistance are achieved using a monolayer blocking layer such as MCO and a hydrogel protective membrane layer on the MCO electrode such as UV-cross-linked polybetaine onto the working electrode, or alternately, polyurethane, polyvinylpyridin or polyvinylimidazole or other membranes. The device also used a porous gold electrode to increase adhesion of the hydrogel on the working electrode, the porous gold formed by repeated electrochemical alloying/dealloying (6000 cycles). The porous gold clearly increases the initial electrode fouling response with greater than or equal to 80% sensor signal loss when tested in serum at 37° C. without membrane protection. This increased fouling may be due to a rougher gold surface having a greater density of blocking layer defects which in turn act as nucleation sites for electrode fouling (as discussed above). Importantly, with addition of the cross-linked polybetaine the fouling is strongly prevented, preserving greater than or equal to 90% of the sensor signal and greater than or equal to 90% of the signal gain initially during the first 2 hours of operation. Optimization of the hydrogel and sensor can preserve at least one of at least greater than or equal to 50% and greater than or equal to 80% of the sensor signal and signal gain for at least one of 3, 4, or 5 days, including formation on gold with reduced porosity and therefore reduced blocking layer defect density. For example, tests with MCO and UV-cross-linked polybetaine and after 3 days of testing at 37° C. in serum results in a signal gain of 105% at 750 μM vancomycin at day 0 and 102% at 750 μM vancomycin at day 3, showing very little loss in signal gain (no significant change within the standard deviation of +/−7%) and no significant change in sensor signal (redox peak current) within the standard deviation. In a preferred embodiment, the sensor provides at least one of less than 30, 20, 10, 5% loss in signal gain after 3 days of operation at greater than or equal to 30° C. in a biofluid such as serum or interstitial fluid. Such longevity of performance is enabled by use of the more energetically stable MCO blocking layer and an optimal gold surface and roughness which otherwise would not be achievable with a blocking layer such as MCH. Furthermore, because the monolayer blocking layer is stable and because robust resistance to fouling is achieved, the electron transfer rates and freedom of movement for the aptamer are preserved, and therefore the frequency response is preserved. Frequency response is heavily dependent on aptamer. In the test shown in FIG. 5D, the vancomycin aptamer was used. However, other aptamers could be used in accordance with the principles of this invention such as, for example, those demonstrated for phenylalanine, cortisol, doxorubicin, irinotecan, or other aptamers. The zero frequency response may shift after 3 days in a biofluid such as, for example, a serum or interstitial fluid by at least one of less than or equal to 5%, less than or equal to 10%, less than or equal to 20, less than or equal to 40, less than or equal to 80%. In one non-limiting example, the zero frequency response for a vancomycin aptamer shifted from 30 Hz to 36 Hz after 3 days (20%). Embodiments of the present invention therefore enable a stable frequency response for zero frequency or for some other measurable signal gain (e.g., 20% gain for 5-50 μM analyte increase). Embodiments of the present invention therefore enable a signal gain that decreases by less than or equal to 2 times, less than or equal to 1.5 times, less than or equal to 1.2 times the original value of the signal gain for at least one of 3, 4, or even 5 days (i.e., would preserve a percentage of the original signal gain greater than or equal to 50%, greater than or equal to 67%, or greater than or equal to 83% of the original total signal gain) at greater than or equal to 30° C. in a biofluid such as serum or interstitial fluid.

With further reference to the present invention, adequate electron transfer then allows for adequate sensor signal at lower square-wave-voltammetry measurement frequencies such that two frequency or similar auto-calibration (calibration free) methods are still possible because a zero-gain frequency that is measurable exists for the sensor. In one embodiment, the sensor may have a zero-gain frequency greater than or equal to 2 Hz, greater than or equal to 5 Hz, greater than or equal to 10 Hz, greater than or equal to 20 Hz, greater than or equal to 50 Hz, or greater than or equal to 100 Hz at greater than or equal to 30° C. in a biofluid such as serum or interstitial fluid, depending on blocking-layer configuration. The zero gain frequency of the tested aptamer may depend on variables such as, for example, thickness of the blocking layer 522, number of defects in the blocking layer 522, and density of defects in the blocking layer 522. For example, if the blocking layer is made too thick or is made with an inadequate amount or density of defects, then the zero gain frequency of the sensor could be at a frequency below 2 Hz which makes accurate measurement more difficult due to a weaker redox current measurement during square wave voltammetry. In one embodiment, the thickness of the blocking layer 522 is less than or equal to 10 nm, more preferably less than or equal to 5 nm, even more preferably less than or equal to 100 A°, and still more preferably less than or equal to 10 A°. In one embodiment, the amount and density of defects is primarily determined by selecting an average slope roughness of the electrode 522 as discussed above. In a further embodiment, the average slope roughness may be less than or equal to at least one of 0.5%, 1%, 2%, 5%, 10%, 20% and 40%. The zero gain frequency of the sensor may also change over time due to factors such as, for example, desorption of at least part of the blocking layer 522, desorption of at least all or some of the at least one aptamer 524, or some combination thereof. In one embodiment, the sensor may exhibit a daily change in zero-gain frequency selected from the group consisting of less than or equal to 10%, less than or equal to 5%, and less than or equal to 2% per day.

### Example 5: Stable Sensor Signal, Energetically Stable Blocking Layer, and Strong Fouling Resistance Via the Surface of the Blocking Layer

With reference to embodiments of the present invention, a stable sensor signal, energetically stable blocking layer, and strong fouling resistance can be achieved using a monolayer blocking layer that is modified with a highly hydrophilic terminal moiety such as a zwitterionic group, polyethylene glycol group, or other suitable anti-fouling group that is highly hydrophilic (e.g., a functional group that creates a layer of bound or adjacent water that repels foulant molecules). Preferably the end-group is a distal moiety with a smaller size than the footprint of the alkyl chain to prevent additional energetically unstable defects from being formed in the self-assembled monolayer, and therefore the moiety occupies a width that at least one of less than or equal to 10 A°, one of less than or equal to 20 A°, or less than or equal to 30 A°, or less than or equal to 50 A°. Another way to specify at preferred size restriction for the distal moieties is that the monolayer of blocking molecules is able to self-assemble in defect-free ordered groups (i.e., a two-dimensional domain) containing at least on average 10 blocking molecules, or at least on average 20 blocking molecules, or more preferably containing at least on average 50 blocking molecules. Preferably such a monolayer blocking layer formed of such molecules provides a fouling resistance of at least one of less than or equal to 5%, less than or equal to 10%, less than or equal to 20%, less than or equal to 40% loss in sensor signal after a time greater than or equal to 3, 4, 5, or more days of operation at a temperature of 37° C., which is significantly greater than a lower limit for a dermal indwelling sensor of greater than or equal to 30° C. The zero frequency response may shift by at least one of less than or equal to 5%, less than or equal to 10%, less than or equal to 20%, less than or equal to 40%, or less than or equal to 80% after a time greater than or equal to 3 days. For example, a vancomycin aptamer only had a zero frequency response shift from 30 Hz to 36 Hz after 3 days (20%). Embodiments of the present invention therefore enable a signal gain that decreases by less than or equal to 2 times, less than or equal to 1.5 times, or less than or equal to 1.2 times the original value of the signal gain for at least one of 3, 4, or even 5 days (i.e., the sensor would preserve greater than or equal to 50%, greater than or equal to 67%, and greater than or equal to 83% of the original total signal gain) at greater than or equal to 30° C. in a biofluid such as serum or interstitial fluid. Preferably each molecule in the blocking monolayer has binding energy that is equal to or more negative than −3.00 eV, or more preferably equal to or more negative than −3.1 eV.

While blocking molecules having an alkyl chain are discussed above, embodiments of the invention may include other polymer chains such as, for example, a polyalkene chain —(CH═CH)ₙ—, a polyethylene glycol chain —(CH₂CH₂O)ₙ—, a polypropylene chain —(CH₂CMeH)ₙ—, or other suitable polymer chains. In one embodiment, the type of polymer chain for the blocking layer molecules is selected based on length of the carbon chain. For example, if the length of the polymer chain is excessive, then electrical response may be undesirably reduced. In one embodiment, for example, blocking layer molecules having, cumulatively in total carbon chain links, an 8, 10, or 12 carbon chain length terminated with phosphatidylcholine may achieve such performance. The assembly of such monolayers may require tight control of the buffer conditions used and may also be aptamer dependent as interactions between the aptamer and or the blocking layer molecules with the blocking layer molecules themselves can strongly impact the quality of the formed sensing monolayer. In another embodiment, the type of polymer chain is selected based on the width of the polymer chain. For example, if the width of the polymer chain is excessive, then bigger gaps between blocking layer molecules (i.e., defects in the blocking layer) may result in solutes reaching the electrode and causing increased oxygen reduction current or increased fouling. In one embodiment, for example, the type of polymer chain is selected so that the width is less than or equal to 50 A°, more preferably less than or equal to 30 A°, more preferably less than or equal to 20 A°, and even more preferably less than or equal to 10 A°.

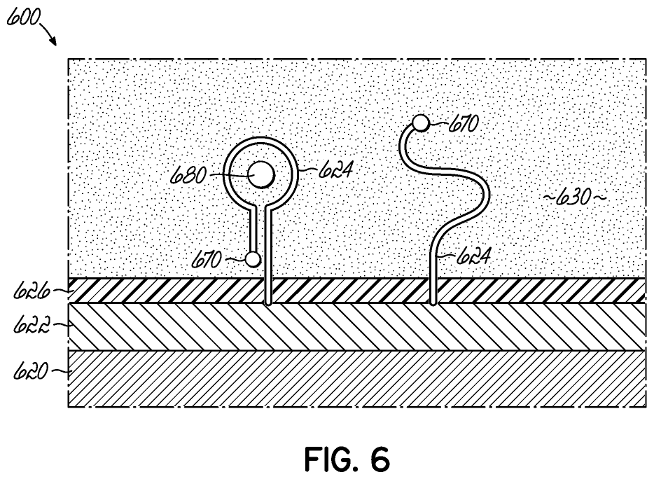

With reference to FIG. 6, where like numerals refer to like features shown and described in FIGS. 1, 3 and 5, an anti-fouling layer 626 may be added to a non-monolayer blocking layer 622. In one embodiment, the non-monolayer blocking layer 622 may include a material where the molecules in the blocking layer are chemically bonded to each other, such as, for example, silicon dioxide (SiO₂), titanium oxide (TiO₂), or other metal or semiconductor oxides with defects such as porosities that mimic the electrochemical performance of monolayer blocking layers as taught herein. As shown in FIG. 6, the anti-fouling layer 626 covers a majority of the non-monolayer blocking layer 622. In one example, non-monolayer blocking layer 622 can be a material with a negatively charged or hydrophilic end group such as, for example, a zwitter ion, polyethyelene glycol, oligo (ethylene glycol), or another suitable end-group that repels foulants and/or protects pores or defects in non-monolayer blocking layer 622 from solutes in fluid 630. In another example, anti-fouling layer 626 could be a self-assembled monolayer of molecules having a molecular structure such as, for example, of R₁—(CH₂)ₙ—R₂, where R₁ is a headgroup that prevents fouling, —(CH₂)ₙ— is a nonpolar alkane chain, and R₂ is an anchoring group that is chemically attached to at least one substrate including the non-monolayer blocking layer 622 and/or the electrode 620. In one embodiment, the head group may be selected for anti-fouling properties. In a further embodiment, the hydrophilic properties of the head group may help prevent or reduce fouling. In one embodiment, the head group may be selected from the group consisting of a hydroxyl group, a zwitterionic group, a polyethylene group, phosphatidycholine group, or other suitable hydrophilic modifiers. In one embodiment, the non-polar alkane chain may be replaced with another polymer chain such as, for example, a polyalkene chain —(CH═CH)ₙ—, a polyethylene glycol chain —(CH₂CH₂O)ₙ—, a polypropylene chain —(CH₂CMeH)ₙ—, or other suitable polymer chains. For different substrates, the selection of the anchoring group, R₂, will be different: a silane (—SiCl₃) anchor group may be used on glass and silicone substrates; and silane or phosphate anchor group may be used on metal oxides substrates. These same attachment techniques may also be used to attach the aptamer 624 to at least one substrate including the non-monolayer blocking layer 622 and/or the electrode 620.

With further reference to embodiments of the present invention, a stable sensor signal, energetically stable blocking layer, and strong fouling resistance can be achieved using a non-monolayer blocking layer that has on its surface a highly hydrophilic end-group such as a zwitterionic group or polyethylene glycol group. Unlike a monolayer blocking layer where packing of the molecules depends on the hydrophilic end-group, a non-monolayer blocking layer is not so limited in choice of the hydrophilic end-group. For example, polyethylene glycol is an excellent antifouling layer when branched, but such branching could exceed an approximately size limit if used at the terminus of an alkylthiol monolayer blocking layer. A non-monolayer blocking layer is not so limited if, for example, the branched polyethylene glycol is silane coupled to a nano-porous SiO₂ blocking layer. In one non-limiting example, the thickness of the SiO₂ blocking layer is greater than or equal to 1 nm and less than or equal to 3 nm. SiO₂ can be deposited by evaporation or sputtering and hydrates with water readily such that it permits electron transfer, while being highly stable compared to most self-assembled monolayer blocking layers. Zwitter ions and peptides are also example suitable anti-fouling moieties that can be silane coupled to the surface of a non-monolayer blocking layer of SiO₂.

With further reference to embodiments of the present invention, a stable sensor signal, energetically stable blocking layer, and strong fouling resistance can be achieved using a monolayer blocking layer that has on its surface a highly hydrophilic end-group such as a zwitterionic group or polyethylene glycol group. In this embodiment the size limit used at the terminus of an alkylthiol monolayer blocking layer may be exceeded, which for an alkylthiol monolayer would energetically destabilize the monolayer and result in poor-stability. Alternately, the molecules in the blocking layer themselves could be energetically unstable as individual molecules in a monolayer as taught in for MCH. This destabilization of the monolayer can be mitigated in one of several ways. First, the monolayer can be chemically bonded between monolayer molecules (cross-linked in part or in full), such hyperbranched polyglycerol with multivalent catecholic bonding groups to an underlying 0.1 to 0.2 nm TiO₂ or SiO₂ film on gold or another suitable electrode. Second, cysteine peptides such as, for example, 5 mer (2 cysteine groups, dithiol) and 7 mer (3 cysteine groups, tri-thiol) peptides can be incubated as a monolayer onto gold. Cysteine residues have a propensity to form a beta-sheet, where the side-chains are on the same side in alternating residues. The peptides can incorporate aspartic acid for the amino acid to improve hydrophilicity due to its low molecular weight. Similarly, zwitter ions, polyethylene glycol, or other suitable terminal moieties can be used but with multiple thiol bonds to the gold electrode. Given the increased stability of a blocking layer where the blocking layer molecules are chemically bonded to each other, the aptamers may also be chemically attached to the blocking layer instead of to the electrode surface. These embodiments taught in this paragraph would provide a stable sensor signal, energetically stable blocking layer, and strong fouling resistance, but some blocking layer molecules with packing density that is less dense than MCH or MCO could also exhibit stable yet strong oxygen reduction current and/or increased electrical capacitance because the blocking layer would not block oxygen diffusion to the electrode surface. Even though this can increase background current (for example MCH data of FIG. 3C) as long as the amount of capacitance and oxygen reduction current remains stable a long-lasting and stable sensor can be achieved. The sensor may exhibit an increase in oxygen reduction current measured at −0.4V compared to a sealed Ag/AgCl reference in serum or buffer solution that may contribute to and increase the background current by at least one of less than or equal to 5%, less than or equal to 10%, or less than or equal to 30% for 3, 4, 5, or more days of operation at a temperature of 37° C. which is significantly greater than a lower limit for a dermal indwelling sensor of greater than or equal to 30° C.

### Example 6: Stable Sensor Signal, Energetically Stable Blocking Layer Enhanced by Continuous Electrochemical Stabilization, and Strong Fouling Resistance

With reference to embodiments of the present invention, measuring sensors by square wave voltammetry or other methods has been previously stated by researchers to destabilize the sensing monolayer (aptamers and/or blocking molecules). The present invention reveals for the first time, that if the sensor is made strongly energetically stable and properly protected from fouling, that a constant or frequent application of electrochemical potential can actually improve stability of the sensor. For example, alkanethiol monolayer desorption may occur via disulfide formation in the monolayer molecules. Oxidation of alkanethiols, such as MCH or MCO, can promote desorption of the MCH and MCO via a similar disulfide formation mechanism. Therefore, in one embodiment of the present invention, a negative voltage may be applied to the sensor working electrode to inhibit oxidation of MCH/MCO to disulfides. However, the increase of background signal arising from the oxygen reduction current and desorption of the blocking layer are undesirable consequences of applying an excessively negative voltage to inhibit oxidation of alkanethiols to prevent disulfide formation. Accordingly, the working electrode may have a negative absolute voltage limit, meaning an applied voltage that is negative, wherein that negative voltage is equal to or more negative than a negative voltage necessary to prevent desorption of the blocking layer molecules via disulfide formation but not equal to or more negative than a negative voltage necessary to cause a significant increase in background signal. For example, in between square wave voltammograms scanned from −0.2 to −0.4 V a DC −0.2 V potential can be held such that the sensor always has an absolute voltage limit that is negative. Alternately, the working electrode may have a negative average voltage wherein the entire range of voltages is negative. For example, the sensor can be scanned continuously over a narrow window with square wave voltammetry, such as −0.2 to −0.35 V such that all voltages applied and the average voltage applied over time to the sensor working electrode are negative. This average negative voltage or absolute negative voltage limit could be applied for at least one of 100%, 90%, 50%, 20%, 10%, 5% or 1% of the time the sensor is in use to prevent monolayer oxidation.

### Example 7: Stable Sensor Signal, Energetically Stable Blocking Layer Enhanced During Incubation, and Strong Fouling Resistance

With reference to embodiments of the present invention, a stable sensor signal, energetically stable blocking layer, and strong fouling resistance with most monolayer blocking layers can only be achieved if the blocking layer is not highly defective. During fabrication of a conventional aptamer sensor constituents may be weakly bound to the sensor surface as indicated by, for example, a portion of the sensing monolayer being physiosorbed to the electrode surface, molecules being incorrectly oriented (e.g., blocking layer that is inverted with thiol facing away from electrode), portions of the electrode being physically or electrochemically fragile, or other aspects of the sensing monolayer that readily degrade or detach from the sensor during initial operation. Weakly bound constituents can be removed before use of the sensor if they are removed during fabrication of the sensor using at least one perturbation method, allowing for further incubation to repair the sensing monolayer as discussed below. The perturbation mechanism may be an electrical mechanism, a mechanical mechanism, a chemical mechanism, a thermal mechanism, or a combination thereof. Particular examples of the perturbation mechanism may include electrical scanning or treatment such as square wave voltammetry, ultrasonic agitation, use of a detergent or other agent, or other suitable techniques. Other methods of removing weakly bonded constituents, may include sonication or ultrasonication, photoactivation with lasers or lamps to add energy locally or to provide heat to activate desorption, heat itself to activate desorption, electron or ion beam stimulated desorption, or other suitable techniques that remove weakly bonded constituents. Perturbation can be used as part of a defect repair process. In one such embodiment, the weakly bound constituents can be removed by applying perturbation during incubation of the blocking layer and aptamers, allowing for subsequent incubation to repair locations where weakly bound constituents were removed. In another such embodiment, perturbation can be applied after incubation to remove weakly bound constituents, followed by a further incubation step to repair locations where weakly bound constituents were removed.

For example, aptamer and MCO incubation under constant perturbation can be performed by cleaning of the electrode followed by aptamer incubation under constant perturbation. In one example, a sensor can be left in a 400 nM cortisol aptamer solution for at least 12 hours while square wave voltammetry is cycled every 27 s at 120 Hz with an amplitude of 35 mV and a step size of 1 mV. Afterwards, the sensors are rinsed in DI water. Then, MCO incubation is performed under constant perturbation for at least 12 hours in a 5 mM solution of MCO in 1×PBS buffer while square wave voltammetry is cycled every 27 s at 120 Hz with an amplitude of 35 mV and a step size of 1 mV.

Furthermore, the plurality of molecules attached to the electrode surface may be either weakly bonded or strongly bonded to the electrode surface. In one embodiment, at least one of less than 40%, less than 20%, less than 10% or less than 5% of plurality of molecules that collectively define the sensing monolayer are weakly bonded to the electrode surface. This can be verified by measuring the sensor operation at 0 hours (initial use before loss of weakly bonded molecules) and after 24 hours where weakly bonded molecules would be removed, using measurement techniques such as, for example, microscopy, electrochemical measurements of oxygen reduction current or redox current, electrical impedance, or other suitable methods used to measure density of a monolayer of molecules on an electrode surface.

With further reference to embodiments of the present invention, during continuous operation of the sensor, embodiments enable a stable signal gain and therefore the sensor accuracy to be maintained over at least 3, 4, or 5 days of operation to within a range less than or equal to +/−60%, less than or equal to +/−40%, or less than or equal to +/−20% accuracy by either direct measurement and/or using a calibration free measurement method such as two-frequency operation.

With further reference to embodiments of the present invention, during continuous operation of the device devices, after at least 3, 4, or 5 days of operation can exhibit an increase in background current that is, in various embodiments less than or equal to 10%, less than or equal to 30%, or less than or equal to 50%.

With further reference to the present invention, during continuous operation of the device after one day of operation, the device can exhibit a change in electron transfer rates (typically measured in ms) that are, in various embodiments, less than less than or equal to 10%, less than or equal to 5%, or less than or equal to 2% per day.

With further reference to the present invention, during continuous operation of the device after one day of operation, the device can exhibit a change in signal response to analyte measured as a percent signal gain, wherein the change in signal response is a value selected from the group consisting of less than or equal to 10% per day, less than or equal to 5% per day, less than or equal to 2% per day, and less than or equal to 1% per day.

With further reference to the present invention, the device has at least one of less than 30, 20, 10, 5% loss in signal gain after 3 days of operation at greater than or equal to 30° C. in a biofluid such as serum or interstitial fluid.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for continuous sensing of at least one analyte in a test fluid, comprising:
an electrode comprising an electrode surface;
a first plurality of molecules, comprising:
a plurality of aptamers comprising attached redox tags, wherein the attached redox tags provide electron transfer with the electrode; and
a blocking layer formed on the electrode, wherein the blocking layer comprises a blocking layer surface and a plurality of features supporting the electron transfer between the electrode and the redox tags; and
wherein the first plurality of molecules, when exposed to temperatures greater than or equal to 30° C., is resistant to at least one of desorption from the electrode and fouling during use of the device for at least 3 days.

2. The device of claim 1, wherein the blocking layer comprises a material having a total binding energy equal to or more negative than −3.05 eV.

3. The device of claim 1, wherein the blocking layer comprises a plurality of mercaptooctanol molecules.

4. The device of claim 1 wherein the blocking layer comprises a plurality of blocking molecules each having a total binding energy that is more negative than −3.1 eV.

5. The device of claim 1 wherein at least one of less than or equal to 40%, less than or equal to 20%, less than or equal to 10% or less than or equal to 5% of the first plurality of molecules are weakly bonded to the electrode surface.

6. The device of claim 1 wherein the electrode is gold and has an average slope roughness selected from the group consisting of at least 0.5%, at least 1%, at least 2%, at least 5%, at least 10%, at least 20%, and at least 40%.

7. The device of claim 1 wherein a majority of the plurality of features in the blocking layer are defects that are less than 0.3 nm in size.

8. The device of claim 7 wherein a majority of the plurality of defects are at least 0.01 nm in size.

9. The device of claim 1, wherein a majority of the plurality of features in the blocking layer are defects, and wherein each of the defects have a fractional area of the surface area of the electrode surface that is a value selected from the group consisting of less than or equal to 0.2, less than or equal to 0.1, less than or equal to 0.05, and less than or equal to 0.02.

10. The device of claim 1, wherein a majority of the plurality of features in the blocking layer are defects, and wherein each of the defects have a fractional area of the surface area of the electrode surface that is a value selected from the group consisting of greater than or equal to 0.001, greater than or equal to 0.002, greater than or equal to 0.005, and greater than or equal to 0.01.

11. The device of claim 1 wherein the blocking layer further comprises a terminus moiety, wherein the terminus moiety reduces fouling, and wherein the terminus moiety has a size selected from the group consisting of less than or equal to 10 A°, less than or equal to 20 A°, less than or equal to 30 A°, and less than or equal to 50 A°.

12. The device of claim 1 wherein the blocking layer further comprises a plurality of blocking molecules having a terminus moiety, wherein the terminus moiety reduces fouling, and wherein the plurality of blocking molecules self-assembles in defect-free ordered groups containing a number of blocking molecules selected from the group consisting of at least 10 blocking molecules, at least 20 blocking molecules, and at least 50 blocking molecules.

13. The device of claim 1, wherein the blocking layer comprises a second plurality of molecules, wherein each of the second plurality of molecules has a terminus moiety, wherein the terminus moiety is a highly hydrophilic end group selected from the group consisting of a hydroxyl group, a phosphatidylcholine group, a zwitterionic group, and a polyethylene glycol group.

14. The device of claim 1, wherein the blocking layer is formed from a metal or semiconductor oxide.

15. The device of claim 14, wherein the blocking layer is silicon dioxide.

16. The device of claim 1, wherein the blocking layer is a monolayer blocking layer.

17. The device of claim 1, wherein the blocking layer is a non-monolayer blocking layer.

18. The device of claim 1, wherein the blocking layer comprises a plurality of blocking molecules which are chemically bonded to each other.

19. The device of claim 1 further comprising an anti-fouling layer, wherein the anti-fouling layer is configured to cover a majority of the blocking layer.

20. The device of claim 19 wherein the anti-fouling layer is formed from a plurality of molecules that are chemically bonded to each other.

21. The device of claim 1, wherein the blocking layer comprises a plurality of amphiphilic molecules, wherein the amphiphilic molecules comprise a head group configured to prevent fouling, a polymer chain, and an anchor group.

22. The device of claim 21, wherein the head group is selected from the group consisting of a zwitterionic group, a polyethylene glycol group, a phosphatidylcholine group, and a hydroxyl group.

23. The device of claim 21, wherein the polymer chain is selected from the group consisting of a polyalkane chain, a polyalkene chain, a polyethylene glycol chain, and a polypropylene chain.

24. The device of claim 1, wherein the blocking layer comprises a plurality of amphiphilic molecules, wherein the amphiphilic molecules comprise a head group configured to prevent fouling, a polymer chain, and an anchor group, wherein the anchor group is bound to a surface selected from the group consisting of the electrode surface or the blocking layer surface, wherein the anchor group is selected based on the surface it is bound to, wherein a pair of the surface and the anchor group is selected from the group consisting of a gold electrode surface and a thiol anchor group, a silver electrode surface and a thiol anchor group, a glass electrode surface and a silane anchor group, a silicon electrode surface and a silane anchor group, a metal oxide blocking layer surface and a silane anchor group, and a metal oxide blocking layer surface and a phosphate anchor group.

25. The device of claim 1 further comprising a protective membrane layer, wherein the protective membrane layer is selected from the group consisting of a hydrogel and a membrane.

26. The device of claim 25, wherein the protective membrane layer comprises a cross-linked polybetaine membrane.

27. The device of claim 1, wherein the device is capable of measuring a plurality of measurements when placed in a test fluid, the plurality of measurements comprising:
a redox current;
a background current;
a signal gain;
a frequency response; and
a sensor signal; and
wherein one or more of the measurements change in response to binding of the analyte with the plurality of aptamers.

28. The device of claim 27 having an initial sensor signal gain when initially placed in the test fluid, the sensor signal gain decreasing by less than or equal to 4 times the initial sensor signal gain.

29. The device of claim 27 further comprising at least one of an anti-fouling layer or protective membrane which preserves greater than or equal to 90% of the sensor signal when compared to an initial sensor signal for greater than or equal to 2 hours of placement in the test fluid.

30. The device of claim 27 further comprising at least one of an anti-fouling layer or protective membrane which preserves at least greater than or equal to 50% or greater than or equal to 80% of the sensor signal and signal gain for a time period selected from the group consisting of at least 3 days, at least 4 days, and at least 5 days.

31. The device of claim 27 wherein the device is further capable of measuring a zero frequency response, and an initial zero frequency response when the device is initially placed in the test fluid, wherein the zero frequency response shifts by a percentage selected from the group consisting of less than or equal to 5%, less than or equal to 10%, less than or equal to 20%, less than or equal to 40%, less than or equal to 80% after a time period selected from the group consisting of at least 3 days, at least 4 days, and at least 5 days.

32. The device of claim 31 wherein the zero-gain frequency is selected from the group consisting of greater than or equal to 2 Hz, greater than or equal to 5 Hz, greater than or equal to 10 Hz, greater than or equal to 20 Hz, greater than or equal to 50 Hz, and greater than or equal to 100 Hz.

33. The device of claim 27 wherein the sensor signal decreases by a percentage selected from the group consisting of less than or equal to 5%, less than or equal to 10%, less than or equal to 20%, and less than or equal to 40% for a time period selected from the group consisting of at least 3 days, at least 4 days, and at least 5 days.

34. The device of claim 27 further capable of measuring an oxygen reduction current at −0.4V compared to a sealed Ag/AgCl reference and an initial oxygen reduction current when initially placed in the test fluid, wherein the oxygen reduction current contributes to and increases the background current by a percentage selected from the group consisting of less than or equal to 5%, less than or equal to 10%, and less than or equal to 30% for a time period selected from the group consisting of at least 3 days, at least 4 days, and at least 5 days.

35. The device of claim 27 further capable of measuring an initial background current when initially placed in the test fluid, the background current increasing by a percentage selected from the group consisting of less than or equal to 10%, less than or equal to 30%, and less than or equal to 50% for a time period selected from the group consisting of at least 3 days, at least 4 days, and at least 5 days.

36. The device of claim 27 having an initial loss of signal of less than 60% when operating in the test fluid for one day, wherein the device is capable of providing sensor operation for at least 4 days.

37. The device of claim 27 wherein the device is capable of implementing two or more frequency calibration free-operation when measuring one or more of the measurements.

38. The device of claim 27 wherein after one day, the background current, increases by a percentage selected from the group consisting of less than or equal to 10% per day, less than or equal to 5% per day, and less than or equal to 2% per day.

39. The device of claim 27 further comprising a zero-gain frequency, wherein the zero-gain frequency is a value selected from the group consisting of greater than or equal to 2 Hz, greater than or equal to 5 Hz, greater than or equal to 10 Hz, greater than or equal to 20 Hz, greater than or equal to 50 Hz, and greater than or equal to 100 Hz.

40. The device of claim 27, wherein after operating for greater than or equal to 4 days, the device has a fouling resistance value selected from the group consisting of greater than or equal to 20%, greater than or equal to 50%, greater than or equal to 75%, and greater than or equal to 90% of the fouling resistance of mercaptooctanol after 24 hours of operation as measured by the amount of signal decrease over time due to fouling.

41. The device of claim 27, further comprising after one day of operation a fouling-induced sensor signal loss that is a value selected from the group consisting of less than or equal to 10% per day, less than or equal to 5% per day, less than or equal to 2% per day, and less than or equal to 1% per day.

42. The device of claim 27, further comprising a sensor accuracy, wherein the sensor accuracy is maintained within a range over 4 days of operation, wherein the range of sensor accuracy is a value selected from the group consisting of less than or equal to +/−60%, less than or equal to +/−40%, and less than or equal to +/−20%.

43. The device of claim 27, wherein after one day of operation the device exhibits a change in electron transfer rates selected from the group consisting of less than or equal to 10% per day, less than or equal to 5% per day, and less than or equal to 2% per day.

44. The device of claim 27 further comprising a zero gain frequency, wherein after one day of operation, the device exhibits a change in zero-gain frequency selected from the group consisting of less than or equal to 10%, less than or equal to 5%, and less than or equal to 2% per day.

45. The device of claim 27, wherein after one day of operation, the device exhibits a change in signal response to analyte measured as a percent signal gain, wherein the change in signal response is a value selected from the group consisting of less than or equal to 10% per day, less than or equal to 5% per day, less than or equal to 2% per day, and less than or equal to 1% per day.

46. The device of claim 27, wherein the device has a loss in signal gain selected from the group consisting of less than or equal to 30%, less than or equal to 20%, less than or equal to 10%, and less than or equal to 5% after 3 days of operation.

47. The device of claim 27 wherein the test fluid is serum.

48. The device of claim 27 wherein the test fluid is interstitial fluid.

49. The device of claim 1, wherein a negative voltage is applied to the electrode, and wherein the electrode has a negative absolute voltage limit.

50. The device of claim 49, wherein the negative absolute voltage limit is applied to the electrode for at least one of 100%, 90%, 50%, 20%, 10%, 5%, or 1% of the time the sensor is in use.

51. The device of claim 1, wherein a negative voltage is applied to the electrode, and wherein the electrode has a negative average voltage.

52. The device of claim 51, where the negative average voltage is applied to the electrode for at least one of 100%, 90%, 50%, 20%, 10%, 5%, or 1% of the time the sensor is in use.

53. A method of providing fouling resistance to an aptamer sensor, the aptamer sensor including an electrode having an electrode surface and an aptamer having a redox tag, the method comprising:
binding a plurality of blocking molecules to the electrode to form a blocking layer having a blocking layer surface,
wherein the blocking layer has a plurality of features supporting electron transfer between the electrode and the redox tag, and wherein the blocking layer and the aptamer are resistant to at least one of desorption from the electrode and fouling during use of the aptamer sensor when exposed to test fluid and temperatures greater than or equal to 30° C. for at least 3 days; and
wherein the aptamer is attached to a surface selected from the group consisting of the electrode surface and the blocking layer surface.

54. The method of claim 53, wherein each of the plurality of blocking molecules include an anchor group.

55. The method of claim 54 wherein the anchor group is a thiol.

56. The method of claim 55, wherein binding the plurality of blocking molecules to form the blocking layer comprises weakly binding a percentage of the plurality of blocking molecules selected from the group consisting of less than or equal to 40%, less than or equal to 20%, less than or equal to 10%, and less than or equal to 5% of the plurality of blocking molecules to the electrode surface.

57. The method of claim 56, wherein the plurality of blocking molecules comprises mercaptooctanol.

58. The method of claim 53 further comprising roughening the electrode surface prior to binding the plurality of blocking layer molecules.

59. The method of claim 58, wherein the electrode surface is roughened to have an average slope roughness of less than or equal to 40%.

60. The method of claim 58, wherein the electrode surface is roughened to have an average slope roughness of less than or equal to 0.5%.

61. The method of claim 58, wherein the electrode surface is mechanically roughened.

62. The method of claim 58, wherein the electrode surface is roughened by depositing a metal onto the electrode surface at a rate of greater than or equal to 10 nm/min.

63. The method of claim 53 further comprising binding at least one of an anti-fouling layer or a protective membrane to the aptamer sensor.

64. The method of claim 63, wherein the protective membrane is placed above the blocking layer surface.

65. The method of claim 63, wherein the anti-fouling layer or protective membrane is bound to the electrode.

66. The method of claim 63, wherein the anti-fouling layer or protective membrane is bound to the blocking layer.

67. The method of claim 63, wherein the anti-fouling layer or protective membrane comprises a second plurality of molecules.

68. The method of claim 67 further comprising crosslinking the second plurality of molecules.

69. The method of claim 68, wherein crosslinking the plurality of molecules comprises applying a crosslinking agent to the second plurality of molecules.

70. The method of claim 68, wherein crosslinking the second plurality of molecules comprises applying UV radiation to the second plurality of molecules.

71. The method of claim 53, wherein each of the blocking molecules comprises a head group configured to prevent fouling, a polymer chain, and an anchor group.

72. The method of claim 71, wherein the head group is selected from the group consisting of a hydroxyl group, a zwitterionic group, and a polyethylene glycol group.

73. The method of claim 71, wherein the polymer chain is selected from the group consisting of a polyalkane chain, a polyalkene chain, a polyethylene glycol chain, and a polypropylene chain.

74. The method of claim 71 wherein binding the plurality of molecules to the electrode to form the blocking layer comprises binding the anchor group to a surface selected from the group consisting of the electrode surface and the blocking layer surface, wherein the anchor group is selected based on the surface it is bound to, wherein a pair of the surface and the anchor group is selected from the group consisting of a gold electrode surface and a thiol anchor group, a silver electrode surface and a thiol anchor group, a glass electrode surface and a silane anchor group, a silicon electrode surface and a silane anchor group, a metal oxide blocking layer surface and a silane anchor group, and a metal oxide blocking layer surface and a phosphate anchor group.

75. The method of claim 63, wherein the protective membrane comprises at least one of a hydrogel or polymer.

76. The method of claim 75, wherein the protective membrane comprises polybetaine.

77. The method of claim 76 further comprising cross-linking the polybetaine by applying at least one of the group consisting of UV radiation and a cross-linking agent.

78. The method of claim 53 further comprising:
using the aptamer sensor for a time; and
applying a negative voltage to the electrode during at least a portion of the time to reduce desorption of at least one of the group consisting of the blocking layer and the aptamer.

79. The method of claim 78, wherein the electrode has a negative absolute voltage limit, the method further comprising applying the negative absolute voltage limit to the electrode for a duration selected from the group consisting of at least one of 100%, 90%, 50%, 20%, 10%, 5%, or 1% of the time.

80. The method of claim 78, wherein the electrode has a negative average voltage, the method further comprising applying the negative average voltage to the electrode for a duration selected from the group consisting of at least one of 100%, 90%, 50%, 20%, 10%, 5%, or 1% of the time.

81. The device of claim 1, wherein the first plurality of molecules, when exposed to temperatures greater than or equal to 30° C. and less than or equal to 47° C., is resistant to at least one of desorption from the electrode and fouling during use of the device for at least 3 days.

82. The method of claim 53, wherein the blocking layer and the aptamer are resistant to at least one of desorption from the electrode and fouling during use of the aptamer sensor when exposed to test fluid and temperatures greater than or equal to 30° C. and less than or equal to 47° C. for at least 3 days.
